(19) United States
(12) Patent Application Publication
(10) Pub. No.: US 2022/0054414 A1
(43) Pub. Date: Feb. 24, 2022

(54) Nanoemulsion Compositions Comprising Saponins for Increasing Bioavailability

(71) Applicant: Readymix Foods Corp., Vaughan (CA)

(72) Inventors: Iraj Mehrnia, Ontario (CA); Thomas Hielscher, Teltow (DE)

(73) Assignee: Readymix Foods Corp., Vaughan (CA)

(21) Appl. No.: 16/997,894

(22) Filed: Aug. 19, 2020

(51) Int. Cl. A61K 9/107; A61K 47/14; A61K 36/67; A61K 47/26; A61K 47/10

(52) U.S. Cl. CPC A61K 9/1075; A61K 47/14; B82Y 5/00; A61K 47/26; A61K 47/10; A61K 36/67

(57) ABSTRACT

Nanoemulsion compositions, and lipid nanoparticle compositions, comprising natural bioactive agents with natural surfactants, such as saponins from the plant Quillaja saponaria and/or phospholipids derived from lecithin. The nanoemulsions are able to increase the bioavailability of the bioactive materials from plant and other sources (e.g. CBD, nutraceuticals, and vitamins). In an embodiment, the composition further comprises Terpenes or Terpenoids, Piperine, and Chitosan. The nanoemulsions are water-soluble and possess a droplet size (e.g. about 43 to 50 nm), which is better or comparable to those made with synthetic surfactants. Furthermore, the nanoemulsions increase the absorption and bioavailability of the bioactive component while decreasing the pre-systemic elimination and first-pass effect of the liver. Methods of making the nanoemulsions comprise sonification: at an energy level of 1000 Ws/g; or without pressure at 2000-2500 Ws/g while recirculating; or applying high pressure (1.5-2.0 bar(g)) while circulating at 1500 Ws/g.

### FIELD OF THE INVENTION

The present disclosure teaches new methods and compositions for delivery of bioactive agents derived from various sources utilizing nanoemulsion and lipid nanoparticle structures. In particular, the present invention is directed to compositions, methods of making and using, the compositions comprising bioactive ingredients from Cannabis (cannabinoids such as CBD, THC, CBN, etc.), Saponins from plants such as Quillaja saponaria, phospholipids (such as lecithin from soy in modified or unmodified form), and other surfactants and co-surfactants used in the food industry.

### BACKGROUND OF THE INVENTION

Delivery of plant extracts, such as cannabinoid, is hindered by: the nature of their chemical structure; and their substantial degradation due to liver first metabolism, thus making the extracts less bioavailable and have a long onset time for a user to experience a biological effect. Most of the plants extracts, e.g. cannabinoids, are insoluble in water and as such poorly distributed. There are many patents and prior art that utilize a variety of surfactants, both natural and synthetic to overcome this obstacle. Synthetic surfactants are able to produce water-soluble emulsions with particle sizes d<100 nm; however, the desire for natural foods and a clean label in the food industry renders them less attractive for health-conscious consumers. Natural surfactants are able to produce water-soluble emulsions; however, the particle sizes will be substantially greater than 100 nm rendering them less bioavailable. For example, Q-Naturale® from Quillaja saponin is one of the natural surfactants that is used for making natural emulsions. This surfactant has previously been shown to be relatively effective at forming stable nanoemulsions with droplet sizes around 200 nm and stable over pH 2 (Y. Yang et al., 2013) [1]. (see the List of References Cited at the end of this disclosure).

Nowadays, there is an increasing demand for bioactive components (e.g.; catechins, phytosterols, curcumin, lycopene, omega-3 fatty acids, and carotenes) due to their various health promoting biological and pharmacological effects including: antioxidant, anticancer, and being protective to chronic disease. Unfortunately, the basic challenges of these useful bioactive compounds are their poor solubility, low bioavailability and instability. Nano-emulsions are known to enhance solubility, stability and bioactivity of various lyphophilic phytochemicals as a result of their small droplet size and high kinetic stability. Ş. Yalçnöz et al. et al. stated that as the particle size of a bioactive compound that is incorporated into a nano-emulsion decreases, the ratio of surface area per unit mass of nano-emulsion increases, which enhances solubility, stability and biological functionality of the bioactive compound by increasing the permeability of it through the biological membranes [2].

Nano-emulsions have been developed to encapsulate a number of different lipophilic components, such as beta-carotene, lycopene, lutein, astaxanthin citral, capsaicin, tributyrin, flavor oil (lemon oil), D-limonene, Vitamin E, omega-3 oil, and alpha-Tocopherol.

Some examples of studies related to increasing bioavailability, solubility and stability of lyphophilic bioactives published in literature are as follows: increasing coenzyme Q10 bioavailability by incorporating coenzyme Q10 into nano-emulsion; increasing stability and oral bioavailability of polyphenols of curcumin and epigallocatechin gallate by incorporation into nano-emulsion; increasing anti-inflammation activity of curcumin by incorporation into nano-emulsion and further improvements with droplet size below 100 nm; increasing bioaccesibility of Vitamin E acetate by incorporation into nano-emulsions as compared to conventional emulsions; increasing bioaccesibility of beta-carotene by nanoencapsulation; increasing oxidative stability of beta-carotene in sodium caseinate stabilized nano-emulsions; and increasing the bioavailability of heptadecanoic acid and Coenzyme Q10 when nanoencapsulated within digestible oil droplets with the smallest size [2].

In all of the examples above, the surfactants used are limited to synthetic surfactants such as: Sucrose monopalmitate, Tween 80, Span 80, Sodium dodecyl sulfate, Polyethylene glycol, lyso-lecithin, Tween 40, Tween 20, sodium caseinate, beta lactoglobulin, Tween20, Span 20, Tween 80, Pluronic F68 and Phospholipid, Sucrose monopalmitate and/or Tween 80, Tween 80 and/or Tween 85, Pluronic F68, etc. In all of the applications noted above, either at least one synthetic surfactant is used, or if a non-synthetic surfactant is used then the resulting size of the nanoemulsion has not been small enough compared to synthetic surfactants. There has not been any prior art available to achieve nanoemulsion with a particle size below 50 nm, which is an important property of nanoemulsions that directly affects its bioavailability. More specifically, there is no known prior art that uses saponin without synthetic surfactant that has a particle size of less than 50 nm. Many reported that achieving particle size of less than 100 nm is not feasible using saponin as surfactant.

According to US patent publication no 20150030748), the emulsifier system consists of at least 5% by weight quillaja saponins, and optionally containing at least one other emulsifier [3]. Despite the high content of saponin in the invention, the smallest particle size noted is greater than 100 nm. In another example, Japanese Publication JP2010142205A describes the use of quillaia extract with a polyoxyethylene Sorbitan fatty acid ester [4]. Other examples are WO 2011/089249, which describes the use of quillaia saponins, plus a substantial proportion of lechitin, as an emulsifier for clear beverages, and EP2359702, which describes the use of quillaia saponins in combination with polymeric emulsifiers for the emulsification of solid, sparingly water-soluble polyphenols, flavonoids and diterpenoid glucosides. In all those inventions there is either a use of synthetic surfactant or particle sizes are not small enough compared to this invention [5], [6].

According to a study conducted by Rao and McClements 2012, nanoemulsion formulation was prepared using Tween 80 and lemon oil; however, the smallest droplet size reported was d=296, 160, 149 and 112 nm for 1×, 3×, 5× and 10× lemon oils [7].

In literature, there is limited studies related to Food-Grade nano-emulsions using natural surfactants with very small particle sizes. Food-Grade nano-emulsions can be safely used for a wide variety of food and beverage compositions, including but not limited to: drinks, beverages, cannabis edibles, cookies, dressings, marinades, sauces, condiments and the like.

The food industry thus needs a natural surfactant for forming and stabilizing emulsions that is safe to consume by a human, and able to form droplets that are very small in diameter. The present invention comprises two natural (i.e. does not comprise synthetic material) small-molecule surfactants, soybean lecithin and quillaja saponin, for producing stable nanoemulsions with particle sizes well below d<100 nm, such as less than 50 nanometers.

### SUMMARY OF THE INVENTION

The present invention comprises nanoemulsions and lipid nanoparticle compositions, and method of making, that include bioactive components, such as but not limited to: cannabinoids, nutraceuticals, bioceuticals, vitamins; and natural surfactants. In an embodiment, the bioactive components comprise one or more of: Saponins and Phospholipids; sugar alcohol; simple polyol, and etc.

According to our disclosure, it has now been established unexpectedly that it is possible to make a nanoemulsion with very small particles (as small as 43 nm) using all natural surfactant(s), namely saponins and phospholipids, and without the need for synthetic surfactants such as Tween 80. This invention therefore teaches a novel method for making a nanoemulsion with a particle size of less than 50 nm (more specifically 43 nm), which comprises a bioactive ingredient(s)/nutraceutical, such as but not limited to cannabinoids in the presence of a natural surfactant systems, which surfactant system consists of Quillaja saponins and phospholipid. More specifically in one embodiment, the surfactant system consists of 2.5% by weight pure saponin from Quillaia saponins and 2.5% by weight phospholipid (enzyme modified lecithin). The Saponins are commercially available as extracts, for example, Quillaia Powder DAB-9 and Q-Naturale™, such extracts containing typically 30% and 14% by weight Saponins, respectively. The quantity of extract used in the compositions of the present invention is calculated to provide the desired target quantity of Saponins. The present invention also can be further enhanced by using Piper nigrum (from pepper family) as a cannabinoid bioavailability and bioefficacy enhancer; and/or by using chitosan (from shells of shrimp and other crustaceans) to improve the composition's bio-absorption.

An object of the present invention is to increase the bioavailability of the bioactive agent in the various administration routes as compared to ones with synthetic emulsions, due to the super small droplet size formed in the nanoemulsions made with only the natural emulsions of the present invention.

This disclosure teaches a product and a process wherein cannabinoids such as CBD, THC and/or other active ingredients associated with cannabis including yet not necessarily limited to cannabidiols, cannabigerol and other medicinal compounds in controlled ways and with specific characteristics, as well as other bioactive ingredients from other sources, are contained or processed into foodstuffs, supplements, medicines, pet foods, and cosmetics.

The production methods used for preparing nanoemulsions and nanoparticle in this invention involves high-energy methods rather than low-energy methods. High energy methods depend on mechanical devices to create powerful disruptive forces for size reduction. Disruptive forces are achieved via ultra-sonicators, microfluidizer and high-pressure homogenizers, which are industrially scalable. The ultra-sonication is the method of choice in this invention due to low instrumental cost and operational cost. Ultrasonication methods depend on high-frequency sound waves (20 kHz and up). They can be used to form a nanoemulsion in situ or reduce size of a pre-formed emulsion. Bench-top sonicators consist of a piezoelectric probe which generates intense disruptive force at its tip when dipped in a sample, ultrasonic waves produce cavitation bubbles which continue to grow until they implode. This implosion sets up shock waves, which in turn create a jet stream of surrounding liquid, pressurizing dispersed droplets and effecting their size reduction. Investigation into operational parameters has revealed that droplet size decreases with increasing sonication time and input power.

The invention teaches a nanoemulsion or nanoparticle composition that employs GRAS ingredients namely saponins, phospholipids (lecithin), and select fatty acids to produce Nano droplets with the average droplet size about 43 nm. The invention also teaches the example products produced by utilizing the nanoemulsion and nanoparticles.

The disclosure teaches a nanoemulsion or nanoparticle composition which can be rendered into several dosage forms, like liquids, creams, sprays, gels, aerosols, foams; and can be administered by equally varying routes like topical, oral, intranasal, pulmonary and ocular.

The invention further teaches a simple and promising nanoemulsion/nanoparticle oral delivery phenomenon and proposes pathways for oral nanoemulsion/nanoparticle absorption from the sublingual mucosa, buccal mucosa, and gastrointestinal tract (GIT). After absorption, nanoemulsion droplets may either enter systemic circulation via hepatic portal vein or alternatively be trafficked into perforated lymphatic endothelium. Active ingredients such as cannabinoids, which enter mesenteric lymph, are directly transported to systemic circulation without undergoing hepatic first pass metabolism.

The invention further teaches a composition that is formulated using optional component, chitosan, as a mucoadhesive biopolymer. In composition, amino groups of chitosan are protonated and the resultant soluble polysaccharide is positively charged (cationic), thus conferring chitosan with mucoadhesive properties. The composition's most attractive property relies on the ability to adhere to mucosal surfaces leading to a prolonged residence time at cannabinoid absorption sites and enabling higher cannabinoid permeation. The composition has further demonstrated capacity to enhance macromolecules epithelial permeation through transient opening of epithelial tight junctions.

Another object of the present invention comprises compositions and methods for making nanoemulsions using natural surfactants, such as but not limited to natural saponins and phospholipids, for delivery of bioactive materials from plant and animal sources (including but not limited to cannabis extracts), which are administered by varying routes such as oral, sublingual mucosa, buccal mucosa, topical, intranasal, pulmonary and ocular. More particularly, this disclosure teaches a novel composition and cannabinoid delivery system that is water-soluble and has a droplet size that is better or comparable to compositions made with synthetic surfactants with particle sizes less than 50 nm. Furthermore, the novel composition in this disclosure increases the absorption and bioavailability of the bioactive component while decreasing the pre-systemic elimination and first-pass effect of the liver.

Another object is to teach a process for making nanoemulsion and nano particle compositions that uses high energy, namely ultrasonication, and alternatively using high shear homogenization and high-pressure homogenization.

Another objective of this invention is to provide a method for making a nanoemulsion and nano particle composition that is sufficiently small in size, more specifically as small as 43 nm in size, that improves absorption and bioavailability either in batch form or in circulation form.

Another objective of this invention is to provide a composition and the method thereof for making nanoemulsion and nano particle composition that is water soluble and it can be incorporated to food and beverage preparations, supplements, medicines, pet foods, skin care products, cosmetics, personal care products and hygiene products.

Another objective of this invention is to provide a composition and production method for nanoemulsion and nano particle composition that includes at least one bioactive component such as but not limited to cannabinoids, nutraceuticals, bioceuticals, vitamins and etc.

Another objective of this invention is to provide a composition, and method of making, comprising a nanoemulsion and nano particle composition that is stable and can be produced in large scale using high energy, namely sonication, and alternatively using high shear homogenization and high pressure homogenization.

Another objective of the present invention is to provide a nanoemulsion and nano particle composition and method thereof that can be incorporated to other compositions by mixing, or directly administered orally (to be absorbed by the gastrointestinal tract), sublingually (to be absorbed by mucosa and buccal mucosa), by inhalation using nebulizers, and by the skin (dermal and epidermal). The nanoemulsion and nano particle composition can be in a form of liquid, solid, or gel and can be dispensed via variety of applicators such as but not limited to droppers, nebulizers, strips and patches, orally dissolving films, creams and lotions, as well as rectal and vaginal suppositories.

A present invention further comprises a nanoemulsion composition and method of making comprising: a) combining a pre-emulsion composition comprising the ingredients of: i) an all natural bioactive compound at about 0.1% wt/wt to about 50% wt/wt, comprising plant and/or food active ingredients without synthetic ingredients comprising: cannabinoids, nutraceuticals, vitamins, or any combination thereof; ii) one or more natural surfactant(s), comprising saponins and/or phospholipids. Then, b) homogenizing said pre-emulsion composition using a high shear mixer about 5 minutes at about 15,000 rpms; c) sonicating the pre-emulsion composition into a nanoemulsion composition using one or more methods comprising: i) a batch sonification at an energy level of 1000 Ws/g; ii) sonification at an energy level of about 2000 Ws/g to about 2500 Ws/g, and without pressure while recirculating the pre-emulsion composition; iii) sonification at an energy level of about 1500 Ws/g, with applying a high pressure of about 1.5 to about 2.0 bar (g), while recirculating the pre-emulsion composition. The resulting composition comprises nanoparticles or nanoemulsions with about 43 nanometers to about 50 nanometers in diameter or droplet size; and wherein said composition is water-soluble and able to increase a bioavailability of the bioactive compound as compared to a composition comprising only synthetic surfactants.

The nanoemulsion composition's natural surfactant comprises up to about 25% wt/wt of powdered saponin extract and/or up to about 50% wt/wt of liquid saponin extract. In an embodiment, the natural surfactant about 2.5% by weight/weight of pure saponin extracted from quillaia Saponins, and/or about 2.5% by weight/weight of a phospholipid comprising derived from enzyme modified lecithin.

In an embodiment, the bioactive compound is cannabinoid at about 0.5% wt/wt to about 5% wt/wt; and the composition further comprises Piper nigrum up to about 0.5% wt/wt and that is able to increase bioavailability and to enhance bioefficacy of said composition.

In an embodiment, the nanoemulsion composition further comprises chitosan derived from shells of shrimp and other crustaceans up to about 5% wt/wt and able to improve said composition's absorption.

In an embodiment, the nanoemulsion composition further comprises a co-surfactant comprising glycerine or sorbital, up to about 5% wt/wt.

In an embodiment, the nanoemulsion composition further comprises a Terpenes and/or Terpenoids up to about 10% wt/wt.

The nanoemulsion compositions of the present invention are formulated into dosage forms comprising, one or more of: like liquids, creams, sprays, gels, aerosols, and foams, or incorporated to other food and beverages (water, alcoholic, and non-alcoholic) products, cosmetics products, pet food products, natural health products, and hygiene products. They can be administered via topical, oral, intranasal, pulmonary, and ocular routes of administration.

Other objects and advantages of the various embodiments of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of this application.

### BRIEF DESCRIPTION OF THE DRAWINGS

Example embodiments will become more fully understood from the detailed description given herein below and the accompanying drawings, wherein like elements are represented by like reference characters, which are given by way of illustration only and thus are not limitative of the example embodiments herein.

FIG. 1 is a graph of nanoemulsion droplet size (diameter on x-axis) versus percent channel to illustrate the droplet size of diluted samples in Series 1 (Sonication in Batch 46 nm) preparations at energy levels of 500 Ws/g, 700 Ws/g, and 1000 Ws/g.

FIG. 2 is a graph of nanoemulsion droplet size versus percent passing that illustrates a percentile droplet size of diluted samples in Series 1 (Sonication in Batch 46 nm) preparations at energy levels of 500 Ws/g, 700 Ws/g, and 1000 Ws/g. After 500 Ws/g, a mean value of 205 nm was reached. This decreased to 63 nm after 700 Ws/g, and 43 nm after 1000 Ws/g (all values from volume distribution).

FIG. 3 are photographs illustrating the appearance of Series 1, 2 and 3 after energy level of 500 Ws/g and 1000 Ws/g. In series 2 sonication was performed in discrete recirculation without pressure. In series 3, a pressure of 1.5 to 2 bar(g) was applied. This resulted in a higher power output, and a higher sonication. Intensity, faster treatment time and also faster temperature increases were observed. Interestingly, after 500 Ws/g, sample 3 that was produced in recirculation under pressure, appeared most translucent. After 1000 Ws/g, a clear difference between sample 2 and 3 was observed, as the sample sonicated without pressure was still more dull. However, sample 3 did not seem to be on the level of the batch sample at this specific energy input.

FIG. 4 illustrates a droplet size of diluted samples Series (1, 2 and 3) after energy level of 500 Ws/g. The optical trend was confirmed by particle size measurement. After 500 Ws/g, sample 3 had the smallest particle size distribution, with an average droplet size of 111 nm. Sample 2 was, with 210 nm, in the range of the batch sample (1: 205 nm).

FIG. 5 illustrates a Percentage Pass of diluted samples Series (1, 2 and 3) at energy level of 500.

FIG. 6 illustrates a droplet size of diluted samples Series (1, 2 and 3) at energy level of 1000. Indeed, after 1000 Ws/g sample 3 had a much smaller droplet size distribution than sample 2, with an average of 67 nm compared to 130 nm. It did not meet the level of sample 1 (43 nm).

FIG. 7 is an illustration of the percentage pass of diluted samples Series (1, 2 and 3) at energy level of 1000.

FIG. 8 is an illustration of droplet size of diluted samples Batch vs. Recirculation without Pressure at different energy levels. To reach the level of the batch sample, 2500 Ws/g had to be invested when sonicating in recirculation without pressure.

FIG. 9 is an illustration of percentage pass of diluted samples Batch vs. Recirculation without Pressure at different energy levels.

FIG. 10 is an illustration of droplet size of diluted samples Batch vs. Recirculation with Pressure at different energy levels. With pressure, 1500 Ws/g were sufficient to reach a mean droplet size of 46 nm. Further sonication changed the droplet size only marginally.

FIG. 11 is an illustration of the Percentage Pass of diluted samples Batch vs. Recirculation with Pressure at different energy levels.

FIG. 12 are photographs illustrating the appearance of Series 2 and 3 with and without pressure at energy level of 1500 Ws/g and 2000 Ws/g. The benefit of pressure was apparent when directly comparing samples at the respective specific energy inputs. The samples sonicated without pressure appeared slightly more dull, whilst those sonicated with pressure were clear and more translucent.

### DETAILED DESCRIPTION OF THE INVENTION

There has thus been outlined, rather broadly, some of the features of the nanoemulsion and nanoparticle composition using saponins and method for increasing bioavailability in order that the detailed description thereof may be better understood, and in order that the present contribution to the art may be better appreciated. There are additional features of the nanoemulsion and nanoparticle composition using saponins and method for increasing bioavailability that will be described hereinafter and that will form the subject matter of the claims appended hereto. In this respect, before explaining at least one embodiment of the nanoemulsion and nanoparticle composition using saponins and method for increasing bioavailability in detail, it is to be understood that the nanoemulsion and nanoparticle composition using saponins and method for increasing bioavailability is not limited in its application to the details of construction or to the arrangements of the components set forth in the following description or illustrated in the drawings. The nanoemulsion and nanoparticle composition using saponins and method for increasing bioavailability is capable of other embodiments and of being practiced and carried out in various ways. Also, it is to be understood that the phraseology and terminology employed herein are for the purpose of the description and should not be regarded as limiting.

The present disclosure is more particularly described in the following examples that are intended as illustrative only since numerous modifications and variations therein will be apparent to those skilled in the art. Various embodiments of the disclosure are now described in detail while referring to the drawings, like numbers, if any, indicate like components throughout the views. Compositions of the present invention comprise: Saponins, Phospholipids (lecithin), Lipids and Fatty Acids, Co-surfactants, Terpenes and Terpenoids, Piperine, and Chitosan. In this disclosure, the applicant successfully utilizes Saponins and Lecithin as natural emulsifiers to create a stable nanoemulsion of fat-soluble compounds including cannabinoids with particle size as small as 43 nm, which is about one-third of the size (120 nm) reported previously according to many prior art disclosures, such as but not limited to a study conducted by Choudhry et al. [8].

### Glossary of Terms

As used herein, the term “nanoemulsions” (also referred to as mini-emulsions, ultrafine emulsions, submicron emulsions) refers to droplet sizes that fall typically in the range of 10-100 nm (and up to 200 nm) and show narrow size distributions. Both oil-in-water (O/W) or water-in-oil (W/O) nano-emulsions can be formed by dispersion or high-energy emulsification methods, as well as by condensation or low-energy emulsification methods (based on the phase transitions that take place during the emulsification process). According to Kumar Gupta et al. 2019, the colloidal delivery systems based on nanoemulsions maybe be utilized in the food and pharmaceutical industries to encapsulate, protect, and deliver lipophilic bioactive components [10]. The small size of the particles in these kinds of delivery systems (r<100 nm) is directly correlated with their rate of bioavailability. The US National Science and Technology Council (2006) defined nanotechnology as matter with dimensions of 1 to 100 nm. Materials that are nanoscale size exhibit physicochemical properties that are different from large particles and that can potentially be used to improve or modify the nutritional, sensorial and structural properties of food products. Particularly, nanotechnology can lead to the advancement of a delivery system for encapsulated bioactive food ingredients or nutraceuticals by enhancing their aqueous solubility, bioavailability and absorption. Consequently, a droplet diameter less than 100 nm is a suitable size range for defining nanoemulsions as having different properties from conventional emulsions. In contrast, microemulsions are thermodynamically stable and contain even smaller particles with a size range of 2-50 nm in diameter.

As used herein, the term “nanoparticle” refers to droplets that are very small particles in the size range 10-100 nm. Nanoparticles can be designed and assembled in diverse structural forms with different physicochemical properties depending on the materials and methods used. These include nano-liposomes, nano-cochleates, micelles, nanoemulsions, solid lipid nanoparticles (SLN) and coacervates (A. Teo et al. 2014) [11]. An advantage of nanoparticles is their ability to remain stable against aggregation and gravitational separation (D. J. McClements et al. 2012) [12]. Also, suspended nanoparticles form transparent or translucent emulsions that can be used in clear beverages without affecting their visual appearance (T. J. Wooster et al. 2008) [13]. In addition, bioactives delivered via nanoparticles have increased bioavailability due to enhanced adsorption and uptake of encapsulated bioactives in the small (L. Hu, Z. Mao et al. 2009) [14]. Most bioactive compounds are poorly soluble in aqueous solution and are sensitive to degradation when exposed to the environmental conditions such as oxygen, light and temperature. The encapsulation of bioactive compounds via nanoparticles, which potentially improves their stability, can lead to the development of new functional foods with the ultimate aim to enhance human health.

It is noted that this invention comprises both nanoemulsion and solid lipid nanoparticle compositions, and their method of making and using. Lipid nanoparticles and nanoemulsions are quite similar in structure except that the lipid cores in nanoemulsions are in a liquid state whereas lipid nanoparticles are in solid state. Polymers, lecithins, and surfactants are used herein as stabilizers.

As used herein, the term “natural surfactant” refers to a surfactant comprising plant and/or animal extracts/surfactants and does not include surfactants synthesized from natural raw materials. As described by Holmberg 2001, the term ‘natural surfactant’ is not unambiguous [9]. Taken strictly, a natural surfactant is a surfactant taken directly from a natural source. The source may be of either plant or animal origin and the product should be obtained by some kind of separation procedure, such as extraction, precipitation or distillation. No organic synthesis should be involved, not even as an after-treatment. There are in fact not many surfactants in use today that fulfil these requirements. Lecithin, obtained either from soybean or from egg yolk, is probably the best example of a truly natural surfactant.

As used herein, the term “bioactive agent” comprises active agents or ingredients in a composition able to produce a physiological effect, and made from bioactive materials either derived from natural sources (such as plant, animal, marine, and/or microbiological sources) or synthesized identical to natural components (nature identical). Nonlimiting examples of bioactive agents for use in the compositions of the present invention comprise: cannabinoids, nutraceuticals, vitamins, phytochemicals, probiotics, fatty acids and etc. These compounds can have poor water solubility, and present low digestion stability and GI absorption, and may be influenced by external environmental conditions (e.g., temperature, light, oxygen, metallic ions, enzymes and water exposure), thus influencing their final performance and purpose.

As used herein, the term “nutraceuticals” refers to biologically active phytochemicals that possess health benefits (P. A. Lachance et al. 2007) [16]. Nutraceuticals may not be essential for maintaining normal human functions, but may enhance human health and wellbeing by inhibiting certain diseases or improving human performance (S. V. Gupta et al. 2019) [17]. Numerous classes of nutraceuticals are found in both natural and processed foods, including carotenoids, flavonoids, curcuminoids, phytosterols, and certain fatty acids. Many of these nutraceuticals have the potential to act as therapeutic agents, and may therefore be suitable for incorporation into functional or medical foods as a means of preventing or treating certain types of cancer. Nutraceuticals vary considerably in their chemical structures, physiochemical properties, and biological effects.

Natural nutraceuticals suitable for use as a natural bioactive agent in the nanoemulsions and lipid nanoparticles of the present invention, comprise by way of non-limiting examples: Soluble Dietary Fibre; Probiotics (Lactobacilli, Gram-positive cocci, Bifidobacteria); Prebiotics (short-chain polysaccharides such as fructose-based oligosaccharides); Polyunsaturated fatty acids (omega-3-(n-3) fatty acids and omega-6-(n-6) fatty acids); Polyphenols (polyphenols such as flavonols, flavones, flavan-3-ols, flavanones and anthocyanins); Spices and Extracts (Most of the spice components are terpenes and other constituents of essential oils).

Other examples of nutraceuticals can be according to the group below based on their pharmacological effects, such as by way of non-limiting examples: 1) Nutraceuticals for treating and/or preventing Alzheimers: β-Carotene, curcumin, lutein, lycopene Antiarthritic: Glucosamine, chondroitin methylsulfonylmethane; 2) Anticancer nutraceuticals: Curcumin, selenium present in fruits and vegetables; 3) Antidiabetic: Ethyl esters of omega-3 fatty acids (docosahexaenoic lipoic acid), dietary fibers; Antioxidant: Ascorbic acid, lycopene, tocopherol; 4) Anti-Parkinson's: Ascorbic acid, creatine; 5) Eye health: Lutein, zeaxanthin; 6) Lipid-lowering agent: Polyunsaturated fatty acids; 7) Inhibition of LDL oxidation: Niacin, green tea, resveratrol, garlic, policosanol, sesame Reduction of total and LDL cholesterol: Plant sterols, flaxseed, garlic, dietary fiber, soy protein HMG-CoA reductase inhibition: Red yeast rice, green tea, garlic, omega-3-fatty acids, plant sterols Reduction of triglycerides: Niacin, red yeast rice, orange juice, flaxseed, resveratrol; 8) Increase in total HDL: Niacin, pomegranate, curcumin; 9) Prevention of cardiovascular diseases: Omega-3 poly unsaturated fatty acids, vitamins, minerals, polyphenols, dietary fibers, flavonoids present in onion, vegetables, grapes, red wine, apples, and cherries; 10) Weight loss: Ephedrine, caffeine, ma huang-guarana, chitosan, green tea, 5-hydroxytryptophan, glucomannan, fenugreek, conjugated linoleic acid, capsaicin, M. charantia; 11) Anti-inflammatory: Cannabis extracts from plants such as Cannabis sativa, Curcumin, Pycnogenol, Capsaicin, Boswellia serrata, and Resveratrol); 12) Natural antidepressant, anti-anxiety, sleeplessness, and relieving the primary symptoms presented with PTSD: St. John's wort, saffron, 5-HTP, SAMe and etc. Additional appropriate plants nutraceuticals for use herein, which have human clinical trial evidence include: Kava kava (Piper methysticum), Chamomile (Chamaemelum nobile), Ginkgo (Ginkgo biloba), Skullcap (Scutellaria laterifolia), Milk Thistle (Silybum marianum), Astragalus (Astragalus membranaceus), Passionflower (Passiflora incarnate), Gotu kola (Centella asiatica), Rhodiola (Rhodiola rosea), Echium (Echium vulgare), Thryallis (Galphimia glauca) and Lemon balm (Melissa officinalis) effective with continued use for the treatment of anxiety.

Vitamins suitable for use as a natural bioactive agent in the nanoemulsions and lipid nanoparticles of the present invention, comprise by way of non-limiting examples: antioxidant vitamins (Vitamins like vitamin E and carotenoids), other fat soluble vitamins (K and D), water soluble vitamins (such as B), and natural antioxidants (Tocopherols, rosemary extract and etc.)

Phytochemicals suitable for use as a natural bioactive agent in the nanoemulsions and lipid nanoparticles of the present invention, comprise by way of non-limiting examples: flavonoids, glucosinolates, organosulfur compounds, saponins, monoterpenes, sesquiterpenes, capsaicinoids, capsinoids and other polyphenolic compounds such as flavonol quercetin and the isoflavones genistein and daidzein, stilbene resveratrol. Examples of Polyphenol-extracts from foods include chocolate, cocoa, black tea, onions, green tea, red wine, grape juice, berries, fruit, and soy. Other phyto-nutraceuticals include glucosamine from ginseng, Omega-3 fatty acids from linseed, Epigallocatechin gallate from green tea, lycopene form tomato etc.

As used herein, the term “cannabinoid” refers to every chemical substance, regardless of structure or origin, that joins the cannabinoid receptors of the body and brain and that have similar effects to those produced by the Cannabis sativa plant. The three types of cannabinoids that people use are recreational, medicinal and synthetic. Research has found that the cannabis plant produces between 80 and 100 cannabinoids and about 300 non-cannabinoid chemicals. The two main cannabinoids are delta-9-tetrahydrocannabinol (THC) and cannabidiol (CBD). The most commonly known of the two is delta-9-tetrahydrocannabinol (THC), which is the chemical that is responsible for the psychoactive effects of cannabis (Alcohol and Drug Foundation 2020) [18].

As used herein, the term “bioavailability” refers to the extent and rate at which the active moiety (bioactive, drug or metabolite) enters systemic circulation, thereby accessing the site of action. Bioavailability of a drug is largely determined by the properties of the dosage form, which depend partly on its design and manufacture. Differences in bioavailability among formulations of a given drug can have clinical significance; thus, knowing whether drug formulations are equivalent is essential (Merck Manuals Professional Edition 2020) [15]. Orally administered compositions with bioactive agents and drugs must pass through the intestinal wall and then the portal circulation to the liver; both are common sites of first-pass metabolism (metabolism that occurs before a drug reaches systemic circulation). Thus, many compositions with bioactive agents and drugs may be metabolized before adequate plasma concentrations are reached. Low bioavailability is most common with oral dosage forms of poorly water-soluble, slowly absorbed compositions with bioactive agents and drugs. Insufficient time for absorption in the GI tract is a common cause of low bioavailability. If the compositions with bioactive agents and drug does not dissolve readily or cannot penetrate the epithelial membrane (e.g., if it is highly ionized and polar), then the time at the absorption site may be insufficient. In such cases, bioavailability tends to be highly variable as well as low.

As used herein, the term “bioefficacy” means the ability of a composition with bioactive agents, drug, or biologic, to produce a desired therapeutic effect independent of potency (amount of the product needed for desired effect).

As used herein the term “Therapeutic Effect” is defined as the result produced by an action—Lack of efficacy/effect is therefore evidence of less than the expected effect of a product refers to a unit of measure to show levels of effectiveness by measuring changes to anatomy and physiology. The term “therapeutic effect” also refers to a therapeutic benefit and/or a prophylactic benefit as described herein. A prophylactic effect includes delaying or eliminating the appearance of a disease or condition, delaying or eliminating the onset of symptoms of a disease or condition, slowing, halting, or reversing the progression of a disease or condition, or any combination thereof.

Saponins

In compositions of the present invention comprises Saponins that are extracted from Quillaia saponaria, or alternatively from Aesculus ippo castanum, Centella asiatica, Ruscus aculeatus, Hedera helix, Terminalia sp., Calendula ocinalis, Soya, Panax Ginseng, lycyrriza sp., Gypsophylla and their aglycons. Saponins are a heterogeneous group of glycosides that are widely distributed in plants [19]. Saponins consist of an aglycone unit linked to one or more carbohydrate chains. The aglycone or sapogenin unit consists of either a sterol, or the more common triterpene unit. In both the steroid and triterpenoid saponins, the carbohydrate sidechain is usually attached to the 3 carbon of the sapogenin.

Saponins possess surface-active or detergent properties because the carbohydrate portion of the molecule is water-soluble, whereas the sapogenin is fat-soluble. The stability and strength of forage saponin foams are affected by pH, and this may have an effect on the development of bloat in ruminants. Saponins are remarkably stable to heat processing, and their biological activity is not reduced by normal cooking.

The presence of saponins has been reported in more than 100 families of plants and in a few marine sources such as star fish and sea cucumber. Triterpene saponins are present in many taxonomic plant groups. In particular, they can be found in parts of dicotyledonous plants (Dicotyledones) such as the seeds of Hippocastani, roots and flowers of Primulae, leaves of Hedrae, roots of Ginseng, bark of Quillaja, roots of Glycyrrbizae, roots of Senegae, leaves of Polygalae amarae, roots of Saponariae, seeds of Glycine max and leaves of Herniariae. Legumes such as soybeans, beans and peas are rich sources of triterpenoid saponins. Steroidal saponins are typically found in members of the Agavaceae, Alliaceae, Asparagaceae, Dioscoreaceae, Liliaceae, Amaryllidaceae, Bromeliaceae, Palmae and Scrophulariaceae families and accumulate in abundance in crop plants such as yams, alliums, asparagus, fenugreek, yucca and ginseng (D. Kregiel et al. 2017) [20].

### Phospholipids (Lecithins)

Phospholipids (lecithin) for use in the present invention comprise by way of non-limiting examples: Lipoid S 75, Lipoid Phospholipon 90 G, Lipoid Phospholipon 90 H, Lipoid S 40, Lipoid S 80, Lipoid E 80, Lipoid Phosal 50 SA, and Lipoid Phosal 53.

Surfactants are amphiphilic molecules which stabilize nanoemulsions by reducing interfacial tension, and prevent droplet aggregation. They tend to rapidly adsorb at oil water interface and provide steric or electrostatic or dual electro-steric stabilization. A common surfactant employed in nanoemulsions is lecithin (phosphatidylcholine) derived from egg yolk or soybean.

Phospholipids have the characteristics of excellent biocompatibility and an especial amphiphilicity. These unique properties make phospholipids most appropriate to be employed as important pharmaceutical excipients, and they have a very wide range of applications in drug delivery systems. Phospholipids are lipids containing phosphorus, a polar portion and non-polar portion in their structures. According to the alcohols contained in the phospholipids, they can be divided into glycerophospholipids and sphingomyelins (J. Li et al. 2015) [21].

Phospholipids are widely distributed in animals and plants, and the main sources include vegetable oils (e.g. soybean, cotton seed, corn, sunflower and rapeseed) and animal tissues (e.g. egg yolk and bovine brain). In terms of production, egg yolk and soybean are the most important sources for phospholipids. However, soybean and egg yolk have differences in the contents and species of phospholipids, mainly including: 1) egg yolk lecithin contains a higher amounts of PC; 2) phospholipids in egg yolk exist long chain polyunsaturated fatty acids of n-6 and n-3 series, primarily arachidonic acid (AA) and docosahexaenoic acid (DHA), which are absent in soybean lecithins; 3) animal lecithins have characteristic of the presence of SM; 4) the saturation level of egg yolk lecithins is higher than that of soybean lecithins, so their oxidative stability is better than that of soybean lecithins; 5) for egg yolk phospholipids, saturated fatty acid is usually at sn-1 position, and unsaturated fatty acid is at sn-2 position, while for soybean lecithin, sn-1 and sn-2 position can be both unsaturated fatty acids. For example, dilinoleoylphosphatidylcholine (DLPC) is the main component of soybean phosphatidylcholine (SPC) (J. Li et al. 2015) [21].

Many synthetic and herbal drugs possess the problem of poor oral bioavailability, and the reason is their very low water solubility or poor permeation through the biological membrane. Poorly soluble drugs have suffered from low bioavailability and inefficacy in therapy due to their low dissolution profile in biological fluid. Without a proper level of drug concentration in the gastrointestinal (GI) fluid, the drugs cannot be effectively transported by the epithelia of the GI tract, resulting in low systemic absorption. However, although most bioactive molecules of plants are biologically polar or water-soluble, they are difficult to pass through the lipid-rich biological membrane and be absorbed by human, the reasons of which include: 1) large molecular weight, and 2) low lipid solubility.

### Lipids and Fatty Acids

Lipids and fatty acids for use in the present invention are preferably from long and medium chain fatty acids, such as oleic acid. Nanoemulsions generally contain 2-20% oil/lipid droplets in case of O/W emulsions, though it may sometimes be significantly larger (up to 70%). Lipids/oils to be used in nanoemulsions are generally propositioned on solubility of final product. Re-esterified fractions derived from soybean oil, sesame oil, cottonseed oil, safflower oil, coconut oil, ricebran oil (labeled as long chain triglycerides (LCT), medium-chain triglycerides (MCT) or short chain triglycerides (SCT) depending on their chain lengths) are used either alone or in combination to formulate nanoemulsions. D-a-Tocopherol (vitamin E) family has been extensively used as a carrier in nanoemulsions. Oleic acid and ethyl oleate have also been used in oral, topical and parenteral nanoemulsions. The type of oil used in a composition of nanoemulsion sometimes determines bioavailable fraction of active component. McClements and Xiao have investigated the influence of formative components and droplet size on the bioavailability of curcumin nanoemulsion (D. J. McClements et al. 2012) [22]. Bio-relevant testing revealed that maximum systemic availability was attained in nanoemulsions made with LCT and MCT, which were digested to an appreciably lesser extent than those made with SCT.

### Co-Surfactants

Sugar alcohols like sorbitol and simple polyols such as glycerol are used in the present invention. Co-surfactants are used to complement surfactants, as they fit suitably in between structurally weaker areas, fortifying the interfacial film. While glycerine and sorbitol are two preferred co-surfactants that are used in this invention, other co-surfactants that are suitable include: propylene glycol, polyethylene glycol, ethanol, transcutol IP, ethylene glycol and propanol.

It is important to note that compositional variables (e.g. oil, presence of other amphiphiles, hydrophilic molecules (i.e. Glycerol, sorbitol) or electrolytes), as well as temperature, may have an influence on hydrophilic and hydrophobic properties and the geometry of the surfactant molecule and the efficiency of a surfactant to generate microemulsion. Sorbitol was chosen as one of the alcohol components in order to improve the solubility. Although sorbitol is almost completely soluble in water, only a negligible amount (max 2 weight %) of oil could be dissolved in these solutions.

### Synthetic Surfactants (Comparison)

For the purpose of comparison, synthetic surfactants, such as but not limited to: Tween 20, Tween 40, Tween 80, Span 20, Span 40 and Span 60, can be used for making nanoemulsions and nanoparticles with the same formulation and method disclosed herein. Gao et al have used Tween 80 and Solutol® HS-15 to develop an orally administered nanoemulsion of Candesartan cilexetil to placate this issue. Developed nanoemulsion increased peak plasma concentration of candesartan cilexetil 27 folds, whereas overall bioavailability increased 10 times in comparison to plain drug suspension (Y. Singh et al. 2017) [23].

### Anti-Oxidants

Anti-oxidants, such as but not limited to Tocopherols and rosemary extract: Emulsified oil and lipids are subject to autoxidation upon exposure to air; many drugs used in nanoemulsion are also highly susceptible to oxidative degradation. Upon oxidation, unsaturated oils give rise to rancidity. If oxidation is to be avoided, then it is common to employ synthetic lipids, which lack the sensitive acyl group. This however is not always feasible, so an extra component namely an antioxidant is added. Antioxidants offer oxidative stability to a formulation by acting either as: 1) a reducing agent, e.g. ascorbic acid, sodium bisulfite, metabisulfite, thiourea and sodium formaldehyde; or 2) a blocking agent, e.g. ascorbic acid esters, butyl hydroxytoluene and tocopherols; or 3) as a synergists, e.g. ascorbic acid, citranoic acid, phosphoric acid, citric acid and tartaric acid. Nanoemulsions are usually transparent, which implies that the entire spectrum of radiation, including visible and UV rays, can easily penetrate oil layers and catalyze photodegradation of drug molecule. Inclusion of chelating agents, pH stabilizers, UV protectants etc. is therefore sometimes required to counter environmental degradation (Y. Singh et al. 2017) [23].

### Preservatives

Optionally preservatives, such as sorbic acid and sorbate, are used in the present invention. Preservatives employed in nanoemulsions should meet criteria like low toxicity, stability to heat and storage, physical and chemical compatibility, reasonable cost, ease of availability, acceptable odor, taste and color, and should have a broad antimicrobial spectrum. Microorganisms thrive in both oil and water, and consequently the selected preservative should attain effective concentration in both the phases. Use of preservatives in parenteral nanoemulsions is more or less avoided due to their toxic potential. Acid and acid derivatives, such as: Benzoic acid, sorbic acid, propionic acid, and dehydro acetic acid, can be used as antifungal agents in composition.

### Terpenes and Terpenoids

Optionally, lemon oil extract can be used in this invention to enhance the viscosity of the dispersed phase as well as for improvement of the sensory characteristics of the nanoemulsions and nanoparticles. Terpenoids (or isoprenoids), a subclass of the prenyllipids (terpenes, prenylquinones, and sterols), represent the oldest group of small molecular products synthesized by plants and are probably the most widespread group of natural products. Terpenoids can be described as modified terpenes, where methyl groups are moved or removed, or oxygen atoms added. Inversely, some authors use the term “terpenes” more broadly, to include the terpenoids.

During the 19th century, chemical works on turpentine led to name “terpene” the hydrocarbons with the general formula C₁₀H₁₆ found in that complex plant product. These terpenes are frequently found in plant essential oils, which contain the “Quinta essentia”, the plant fragrance.

Citral is one of the most important flavor compounds that is widely used in the food and beverage industries. Citral is chemically unstable and degrades over time in an acidic environment; and, it also can be affected by heat, light and oxygen (C.-P. Liang et al. 2004) [24]. Citral and limonene are the major flavor components of citrus oils. Citral (3,7-dimethyl-2,6-octadienal) and limonene [1-methyl-4-(1-methylethenyl)cyclohexene] are two of the most important flavor compounds in essential oils obtained from citrus fruits. Citral consists of neral and geranial, which are geometrical isomers. The relative viscosities of the two phases: the dispersed (η_{d}), and the continuous phase (η_{c}), has a strong influence on the outcome of the size reduction process.

When relative viscosity is too high, droplets become resistant to breakup, and instead start rotating upon their own axis when subjected to shear. The oil type and oil volume fraction also affect the droplet size. When dealing with very thick oils, droplet size can be reduced by raising the viscosity of the continuous phase. Considering factors described above, it is apparent that the final droplet size attained is a complex interplay between surfactant chemistry, applied shear, etc. This interplay must be taken into account whilst selecting or optimizing the process for manufacturing nanoemulsions. Using citral can affect the relative viscosities of the dispersed phase to further help reduce the nanoemulsion particle size (Y. Singh et al. 2017) [23].

The main constituents in single-fold lemon oil used in the present invention are monoterpenes (>90%), whereas the major constituents in 10-fold lemon oil are monoterpenes (about 35%), sesquiterpenes (about 14%) and oxygenates (about 33%). The density, interfacial tension, viscosity, and refractive index of the lemon oils used herein increased as the oil fold increased (i.e., 1×<3×<5×<10×).

The stability of oil-in-water emulsions formed by homogenizing lemon oil with an aqueous surfactant solution depended strongly on the lemon oil fold: the stability to droplet growth increased as the oil fold increased. This effect is attributed to changes in the overall water-solubility profiles of the lemon oils with oil fold, since this would be expected to influence the rate of droplet growth caused by Ostwald ripening. The presence of relatively high levels of lemon oil constituents with low water-solubility in high fold oils (10×) may have been able to inhibit droplet growth by generating a compositional ripening effect that opposed the Ostwald ripening effect. This will be useful for designing stable nanoemulsion preparation using saponins (J. Rao et al. 2012) [7].

### Piperine

Optionally, piperine from black pepper extract is used in this invention to enhance the bioavailability of the nanoemulsion and nanoparticles. Piperine is extracted from black pepper by using dichloromethane. Aqueous hydrotropes can be used in the extraction to result in high yield and selectivity. The amount of piperine varies from 1-2% in long pepper, to 5-10% in commercial white and black peppers. Further, it may be prepared by treating the solvent-free residue from an alcoholic extract of black pepper, with a solution of potassium hydroxide to remove resin (said to contain chavicine, an isomer of piperine). And a solution of the washed, insoluble residue in warm alcohol, from which the alkaloid crystallizes on cooling. Certain excipients, such as tocopheryl polyethylene glycol 1000 succinate (TPGS) and Labrasol® that is used in formulating nanoemulsions, have the unique ability of inhibiting ATP dependent pglycoprotein (P-gp) transporter and have been exploited to increase oral bioavailability of poorly soluble anticancer drugs like Paclitaxel. As an alternative and with the intent of using natural ingredients, this invention uses piperine as an agent to improve the bioavailability of the composition as an optional element (Y. Singh et al. 2017) [23].

### Chitosan

Optionally, chitosan from marine sources is used in this invention. Chitosan coated nanoemulsions have been grafted for enhancing oral protein absorption by exploiting the mucoadhesive nature of the polymer, which enhances residence time of nanoemulsion droplet at the absorptive site.

### Fate of Nanoemulsion: In Vivo

Upon oral administration, nanoemulsions enter the gastrointestinal tract (GI tract) and are subjected to variety of environmental conditions. Persson et al. (2006) have come up with a theory that postprandial response is stimulated at least partially in such cases [25]. Stimulation of the ‘lipidsensing’ mechanism in the GI tract leads to the secretion of gastric lipases, which start fractional digestion of LCT or MCT making up the nanoemulsion, to yield simpler di-glycerides, mono-glycerides and free fatty acids (Y. Singh et al. 2017) [23].

The small size of nanoemulsion droplets accelerates this lipase activity. Digestion of the oily component frees up the drug, which usually undergoes nanoprecipitation. In other instances, the drug may just partition out of the oil droplet into the surrounding aqueous environment. Products in the GI tract stimulate secretion of bile and delay GI tract motility. Components of bile aid in solubilization of nanoemulsions. By acting as endogenous surfactants and may form colloidal structures known as mixed micelles. Bile and preexisting mixed micelles further solubilize free drug and carry it across aqueous unstirred diffusion layer for absorption (Y. Singh et al. 2017) [23].

Nanoemulsion droplets are sometimes absorbed intact via paracellular or transcellular pathways, or via M-cells present in Peyer's patches. Additionally, collisional absorption also occurs, which involves accidental impact absorption of nanoemulsion droplet. Due to the flexible nature of droplets, nanoemulsions tend to stick and squeeze through the absorption barrier, bending and changing their contours according to gaps available in the packed bilayer.

After absorption, nanoemulsion droplets may either enter the systemic circulation via the hepatic portal vein, or alternatively be trafficked into perforated lymphatic endothelium. Drugs which enter mesenteric lymph are directly transported to systemic circulation without undergoing hepatic first pass metabolism. Therefore, numerous mechanisms work in unison to offer several pathways which alter oral bioavailability of poorly available drugs when they are administered via nanoemulsion topically.

It is a challenge to enhance permeation of several drugs intended for topical application. These are limited by poor dispersibility in topical vehicles like gels, creams, patches or possess skin irritant action. Nanoemulsions have been explored for topical uptake of such drugs. They provide a combination of penetration enhancement (by altering lipid bilayers) and concentration gradient by acting as tiny reservoirs of drugs. For instance, a nanoemulsion (made of soybean lecithin, tween and poloxamer) containing menthol, methyl salicylate and camphor was prepared by high energy method and incorporated in a hydrogel. The resulting composition had high permeation rates. Nanoemulsions can be employed to deliver small molecules systemically via a topical route. In an illustrative study, an O/W nanoemulsion (made with high pressure homogenization using soybean oil, phosphatidyl choline, Tween 80) containing a, d or alpha-tocopherol was compared with their respective nanosuspensions. It was observed that systemic bioavailability along with antioxidant activity of δ and γ tocopherol increased 2.5 times when they were delivered as nanoemulsion (F. Kuo et al. 2008) [26]

### Routes of Administration

This disclosure teaches how to increase bioavailability from various administration routes due to super small droplet size of the resulting nanoemulsion and the method of administering the nanoemulsion through one or more routes of: Oral route, Sublingual and buccal routes, Rectal route, Vaginal route, Nasal route, Cutaneous route, and Transdermal route.

### Oral, Sublingual, and Buccal Drug Delivery:

Amongst the various routes of drug delivery, the oral route is perhaps the most preferred to the patient and the clinician alike. However, peroral administration of drugs has disadvantages such as hepatic first pass metabolism and enzymatic degradation within the GI tract that prohibit oral administration of certain classes of drugs, especially peptides and proteins. Consequently, other absorptive mucosae are considered as potential sites for drug administration. Transmucosal routes of drug delivery (i.e., the mucosal linings of the nasal, rectal, vaginal, ocular, and oral cavity) offer distinct advantages over peroral administration for systemic drug delivery. These advantages include possible bypass of first pass effect, avoidance of pre-systemic elimination within the GI tract, and, depending on the particular drug, a better enzymatic flora for drug absorption (Shojaei et al. 1998) [27].

The oral cavity, nonetheless, is highly acceptable by patients, the mucosa is relatively permeable with a rich blood supply, it is robust and shows short recovery times after stress or damage, and the virtual lack of Langerhans cells makes the oral mucosa tolerant to potential allergens. Furthermore, oral transmucosal drug delivery bypasses first pass effect and avoids pre-systemic elimination in the GI tract. These factors make the oral mucosal cavity a very attractive and feasible site for systemic drug delivery. Within the oral mucosal cavity, delivery of drugs is classified into three categories: (i) sublingual delivery, which is systemic delivery of drugs through the mucosal membranes lining the floor of the mouth, (ii) buccal delivery, which is drug administration through the mucosal membranes lining the cheeks (buccal mucosa), and (iii) local delivery, which is drug delivery into the oral cavity.

The oral mucosae in general is a somewhat leaky epithelia intermediate between that of the epidermis and intestinal mucosa. It is estimated that the permeability of the buccal mucosa is 4-4000 times greater than that of the skin (W. R. Galey et al. 1976) [28]. As indicative by the wide range in this reported value, there are considerable differences in permeability between different regions of the oral cavity because of the diverse structures and functions of the different oral mucosae. In general, the permeabilities of the oral mucosae decrease in the order of sublingual greater than buccal, and buccal greater than palatal (D. Harris et a. 1992) [29].

There are two permeation pathways for passive drug transport across the oral mucosa: paracellular and transcellular routes. Permeants can use these two routes simultaneously, but one route is usually preferred over the other depending on the physicochemical properties of the diffusant. Since the intercellular spaces and cytoplasm are hydrophilic in character, lipophilic compounds would have low solubilities in this environment. The cell membrane, however, is rather lipophilic in nature and hydrophilic solutes will have difficulty permeating through the cell membrane due to a low partition coefficient. Therefore, the intercellular spaces pose as the major barrier to permeation of lipophilic compounds and the cell membrane acts as the major transport barrier for hydrophilic compounds. Since the oral epithelium is stratified, solute permeation may involve a combination of these two routes. The route that predominates, however, is generally the one that provides the least amount of hindrance to passage.

The buccal mucosa offers several advantages for controlled drug delivery for extended periods of time. The mucosa is well supplied with both vascular and lymphatic drainage and first-pass metabolism in the liver and pre-systemic elimination in the gastrointestinal tract are avoided. The area is well suited for a retentive device and appears to be acceptable to the patient. With the right dosage form design and formulation, the permeability and the local environment of the mucosa can be controlled and manipulated in order to accommodate drug permeation. Buccal drug delivery is a promising area for continued research with the aim of systemic delivery of orally inefficient drugs as well as a feasible and attractive alternative for non-invasive delivery of potent peptide and protein drug molecules. However, the need for safe and effective buccal permeation/absorption enhancers is a crucial component for a prospective future in the area of buccal drug delivery (Shojaei et al. 1998) [27]

### Rectal Route:

Rectal drug delivery is an efficient alternate to oral and parenteral route of administration in partial avoidance of first pass metabolism and protein peptide drug delivery. This route allows both local and systemic therapy of drugs. Controlled absorption enhancement of drugs can be achieved by the rectal route because of the constant conditions in the rectal environment. In the present review various absorption enhancers with their mechanism of action in improving drug absorption through rectal epithelium and the potential of rectal route in delivering protein and peptides, analgesics and antiepileptics are discussed (Lakshmi Prasanna J. et al. 2012) [30].

### Vaginal Route:

Although clinicians commonly use topically administered drugs in the vagina, this route for systemic drug administration is somewhat novel. Experience with a variety of products demonstrates that the vagina is a highly effective site for drug delivery, particularly in women's health. The vagina is often an ideal route for drug administration because it allows for the administration of lower doses, steady drug levels, and less frequent administration than the oral route. With vaginal drug administration, absorption is unaffected by gastrointestinal disturbances, there is no first-pass effect, and use is discreet (N. J. Alexander et al. 2004) [31]

### Nasal Drug Delivery:

Nasal drug administration has been used as an alternative route for the systemic availability of drugs restricted to intravenous administration. This is due to the large surface area, porous endothelial membrane, high total blood flow, the avoidance of first-pass metabolism, and ready accessibility. The nasal administration of drugs, including numerous compound, peptide and protein drugs, for systemic medication has been widely investigated in recent years. Drugs are cleared rapidly from the nasal cavity after intranasal administration, resulting in rapid systemic drug absorption. Several approaches are here discussed for increasing the residence time of drug formulations in the nasal cavity, resulting in improved nasal drug absorption (S. Türker et al. 2004) [32]

A major hurdle in targeting the brain is the presence of the blood brain barrier (BBB). It restricts entry of hydrophilic and high molecular weight molecules like peptides. However, olfactory vein in nasal mucosa provides a direct passage between nose and brain. This has been exploited by use of nanoemulsions loaded with anti-Alzheimer's, anti-parkinsonism, antipsychotic drugs for targeting brain. Risperidone, an antipsychotic, exhibits low bioavailability due to extensive first pass metabolism. This warrants administration of huge doses, which brings about numerous side effects. To reach the brain in effective concentrations and to avoid any unnecessary side effects a strategy involving nanoemulsion has been implemented; that improves bioavailability by preventing first pass metabolism and facilitating blood-brain barrier transport. Risperidone was dissolved in capmul MCM, tween 80, transcutol and propylene glycol to (48%, w/w) to form an O/W nanoemulsion spontaneously. Ultra-fine globule size of the developed nanoemulsion (15.5-16.7 nm) ensured quick and effective risperidone delivery to brain following intranasal administration (M. Kumar et al. 2008) [33].

Selection of a surfactant/surfactant blend not only influences size and stability of nanoemulsion; but, it sometimes also determines its toxicity, pharmacokinetics and pharmacodynamics. As disclosed in U.S. Pat. No. 9,925,149 B2 by Kaufman, 2018, “a smaller nanoparticle size (less than 60 nm), and a natural lipid and phospholipid nanoparticle composition (that mimics a plasma lipoprotein), can avoid extensive pre-systemic metabolism, avoid uptake by the reticulo-endothelial system of the liver and spleen as a foreign substance, and prevent premature clearance from the body” [34].

Tween 20, 40, 60 and 80 (polyoxyethylene sorbitan monolaurate); Span 20, 40, 60 and 80 (Sorbitan monolaurate); and Solutol HS-15 (polyoxyethylene-660-hydroxystearate) are all synthetic surfactants regularly used for making nanoemulsions with or without the use of phospholipids. Saponins, on the other hand, are natural surfactants from a plant source with the limitation in their ability to create small nanoemulsions with particle sizes normally above 100 nm.

The process of making nanoemulsion and nanoparticles in this invention uses high energy that can be accomplished by sonication, and alternatively by high shear homogenization or high pressure homogenization, or the combination of one or more of the above mentioned approaches with varying degrees of pressure, temperature, and energy level with or without the use of solvents and processing aids for making small particles.

The method utilized in this invention for making nanoemulsion/nano particle uses saponin and sonication to achieve particle size as small 43 nm using sonication which has not reported feasible in the prior art.

In one example, this invention teaches how to make a lipid nanoemulsion that has a particle size of d<43 nm using saponins and phospholipids as natural surfactants.

High energy methods depend on mechanical devices to create powerful disruptive forces for size reduction. Disruptive forces are achieved via ultrasonicators, microfluidizer and high pressure homogenizers which are industrially scalable. Their versatility lies in the fact that almost any oil can be subjected to nanoemulsification. A microfluidizer (Microfluidics™ Inc., U.S.A) concomitantly uses hydraulic shear, impact, attrition, impingement, intense turbulence and cavitation, to effect size reduction. It forces feed material through an interaction chamber consisting of microchannels under influence of a high-pressure displacement pump (500-50,000 psi), resulting in very fine droplets.

Piston gap homogenizers work on principle of colloid mills. A coarse emulsion is made to pass through a narrow gap (of dimension b 10 μm) between a fixed stator and a rapidly moving rotor. Size reduction is caused by high shear, stress and grinding forces generated between rotor and stator. The upper ceiling of droplet size can be ascertained by fixing dissipation gap to required size, which implies that a yield will not be obtained unless and until emulsion is ground down to a size which is equal or lower to that of the gap between rotor and stator.

Ultrasonication methods depend on high-frequency sound waves (20 kHz and up). They can be used to form and nanoemulsion in situ or reduce size of a pre-formed emulsion. Bench-top sonicators consist of a piezoelectric probe which generates intense disruptive force at its tip. When dipped in a sample, ultrasonic waves produce cavitation bubbles which continue to grow until they implode. This implosion sets up shock waves, which in turn create a jet stream of surrounding liquid, pressurizing dispersed droplets and effecting their size reduction. Investigation into operational parameters has revealed that droplet size decreases with increasing sonication time and input power. Probes in an ultrasonicator are available in variety of dimensions which affect their functionality. Usually narrower probes are preferred for working on small volume batches. Relative placement of probe in the sample, i.e. depth to which it is dipped alters pattern of wave reflection and pressure distribution and consequently it should not touch any solid surface. Procedurally, a coarse emulsion is prepared by addition of a homogenous oil phase to aqueous phase under mechanical stirring. The emulsion is then subjected to ultrasonication at different amplitudes for short time cycles until desired properties are obtained for nanoemulsion.

Adherence to a strict droplet size is a perquisite whilst fabricating nanoemulsions, and size estimation is mandatorily performed following composition. Droplet size influences many properties. Larger, more spherical drops will typically flow easier than smaller or distorted droplets, which tend to stick together. Uniformity of droplet size distribution is measured by polydispersity index; nanoemulsions are generally referred to as ‘monodisperse’ if polydispersity index is <0.2. Particle size analyzers measure droplet radius using photon correlation spectroscopy (PCS) or laser diffraction. PCS has limitations though, in terms of overall derivable information. It sometimes misses out on smaller populations, which differ substantially from average population. It is also impossible to differentiate blank droplets (which do not possess any drug molecule), surfactant aggregates, liposomes, micelles, nanoparticles or one colloidal form from other. Additionally, shape of oil droplets is taken as a perfect sphere which is not always the case. Furthermore, dilution of a sample is often required, which alters its native state. Therefore, for exact visualization (globule size, volume fraction, shape,) electron microscopy (SEM, TEM, cryo-TEM, freeze-fracture), neutron and X-ray scattering are applied to substantiate data obtained via PCS. SEM produces considerably deep two-dimensional images and is beneficial in identifying topography, contours and morphology of a droplet.

Zeta Potential is used for gauging charge on nanoemulsion surface, which provides clues towards its long-term stability and in some cases interaction with the target matrix. It is determined indirectly using principle of electrophoretic mobility. As a rule of thumb zeta potential values >+30 mV or <−30 mV are considered as good indicators of Long thermostability. Nanoemulsions with lower zeta potential may eventually aggregate and even phase separate. Manipulating zeta potential is therefore a method of enhancing emulsion stability.

### EXAMPLES

The following examples are provided for illustrative purposes only and are not intended to limit the scope of the invention. The figures illustrate an example embodiment comprising bioactive component such as but not limited to Cannabidiol (CBD), and with phospholipid, sugar alcohol, and simple polyol which are selected from the group outlined below.

### Example 1

### Preparation of Base Nanoemulsion

The Basic Nanoemulsion Composition Formulation and method of making is comprised of the following.

1. 1 Materials.—The various embodiments of the present invention comprise the following ingredients:

TABLE OF INGREDIENTS

[A] Bioactive/Nutraceutical Agents, such as CBD Isolate Powder 0.1% to
50%.
[B] Bioavailability and bioefficacy enhancer such as Piperine (BioPerine ®
BLACK PEPPER EXTRACT) 0% to 0.5%.
[C] Lipids such as OLEIC SUNFLOWER OIL (GROUPE EMILE, FRANCE)
0% to 50%.
[D] Terpenes and Terpenoids such as OIL LEMON TYPE 10-FOLD
EXTRA (CITRUS AND ALLIED ESSENCES LTD.) 0% to 10%.
[E] Powdered Saponin Extracts such as Saponin DAB 9 (Base Quillaja)
(DR. H. SCHMITTMANN GMBH) 0% to 25%.
[F] Liquid Saponin Extracts such as Q-NATURALE ® Quillaja Extract
(Ingredion, USA) 0% to 50%.
[G] Phospholipids such as Alcolec LEM (Enzyme-Modified Soy Lecithin,
American Lecithin Company) 0.5% to 20%.
[H] Antioxidants such as Tocopherol Mix (Fortium MTD10, Kemin Food
Technologies) 0% to %3.
[I] Solvents such as Vegetable Glycerin (Now Foods) 0% to 35%.
[J] Sugar alcohols such as Crystalline Sorbitol (Caldic Canada) 0% to
35%.
[K] Synthetic surfactants such as Tween 80 (Croda Canada Ltd) 0% to
25%.
[L] Natural polymers such as Chitosan (Tidal Vision, USA) 0% to 5%
[M] Preservatives such as Potassium Sorbate 0% to 3%
[N] Water as required 0% to 99%

A setup for Batch and Circulation equipment for use in the present invention comprises: UP400St with S242d22D, and Recirculation Setup (comprising an UIP2000hdT, equipped with an cascatrode CS4d40L1 and a booster B4-1.8 (up) in a flow cell FC100L1K-1S) and a Discrete Recirculation Vessel (the setup included separate vessel to catch the sonicated material to avoid mixing with unsonicated liquid).

To set a base level, 200 g of the emulsion-premix were sonicated in batch with the UP400St and Sonotrode S24d22D (100% amplitude=46 μm). In recirculation, an UIP2000hdT, equipped with a Cascatrode CS4d40L1 and a booster B4-1.8 (up) in a flow cell FC100L1K-1S, was used. This constellation yielded an amplitude of 54 μm. The setup included a progressive cavity pump MD012-12 (Seepex), Pressure- and thermo-sensor, pressure valve and cooling tubes. The trials were conducted as a Discrete Recirculation, which means the sonicated material was collected in a separate vessel to avoid mixing with unsonicated liquid.

### 1. 2 Method for Preparation of the Nanoemulsion

A nanoemulsion with a composition given in Table 1 was prepared by blending the following.

TABLE 1

| Component | % wt/wt |
|---|---|
| CBD Isolate Powder (99% pure) | 0.5%-5% |
| Organic Deodorized Oleic Sunflower Oil | 0.5%-5% |
| Oil Lemon Type 10-fold Extra | 0%-2% |
| Saponin DAB 9 (Base Quillaja) | 2%-10% |
| Alcolec LEM (Enzyme-Modified Soy Lecithin) | 2%-5% |
| Tocopherol Mix (Fortium MTD10) | 0%-0.5% |
| Vegetable Glycerin | 0%-30% |
| Crystalline Sorbitol | 0%-30% |
| Deionized water | To 100% |

Part (I): Consisting of the step of combining one-third the amount of deionized water, crystalline sorbitol and glycerine until a clear solution was achieved under mild heating (alternatively liquid sorbitol can be used by adjusting the amount of deionized water required accordingly).
Part (II): Consisting of the step of separately, two-thirds the amount of deionized water was added to Quillaia extract powder and it was blended gently to minimize foaming.
Part (III): Part (II) was added to Part (I) and the blend was stirred gently until homogeneous.
Part (IV): Oil soluble bio-active ingredient(s), CBD isolate powder in this example, was added to carrier oil (sunflower oil) and blended under mild heat until completely dissolved. Next, phospholipid, Alcolec LEM in this example, was added to this mix and blended gently until homogenous.
Part (V): Part (IV) was homogenized using high shear mixer (Polytron PT3100) at a speed of 15,000 rpm for 5 min to make the pre-emulsion composition. The resulting coarse emulsion was sonicated in one of the following systems:
i. Series (1) in batch with the Hielscher Ultrasonics GmbH UP400St and its sonotrode S24d22D (100% amplitude=46 μm). Series (2) without pressure in recirculation, an UIP2000hdT, equipped with cascatrode CS4d40L1 and a booster B4-1.8 (up) in a flow cell FC100L1K-1S (recirculation included a separate vessel to catch the sonicated material, to avoid mixing with unsonicated liquid).
ii. Series (3) with pressure in recirculation, an UIP2000hdT, equipped with cascatrode CS4d40L1 and a booster B4-1.8 (up) in a flow cell FC100L1K-1S1S (recirculation included separate vessel to catch the sonicated material, to avoid mixing with unsonicated liquid).

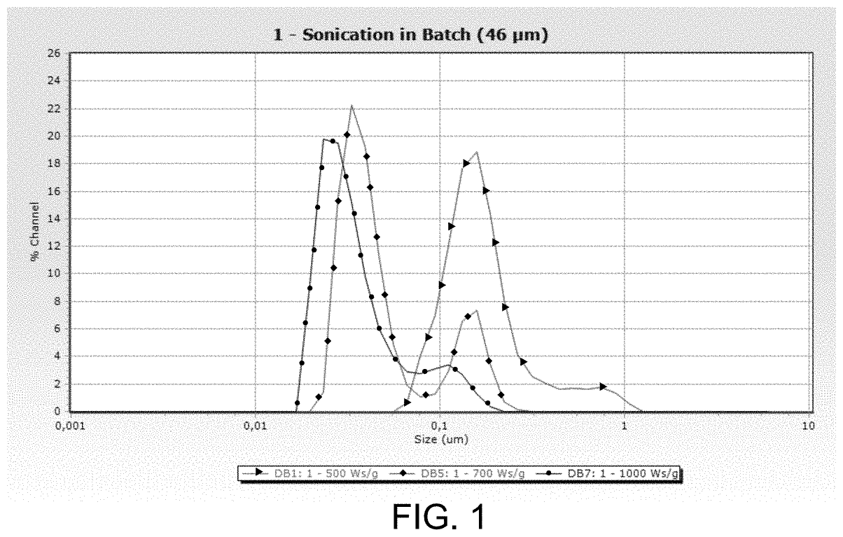

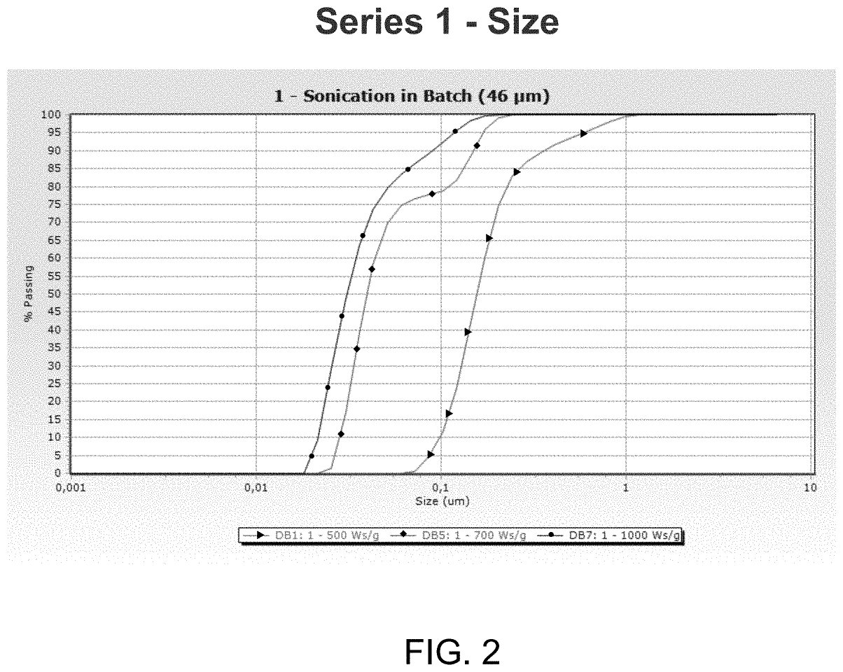

See FIG. 1 illustrates a nanoemulsion droplet size of 46 nm (or 0.046 μm) (black line peak) achieved with a sonification at 1000 Ws/g. And FIG. 2 illustrates a percentile droplet size of diluted samples in Series 1 (Sonication in Batch 46 nm) preparations at energy levels of 500 Ws/g, 700 Ws/g, and 1000 Ws/g. After 500 Ws/g, a mean value of 205 nm was reached. This decreased to 63 nm after 700 Ws/g, and 43 nm after 1000 Ws/g (all values from volume distribution).

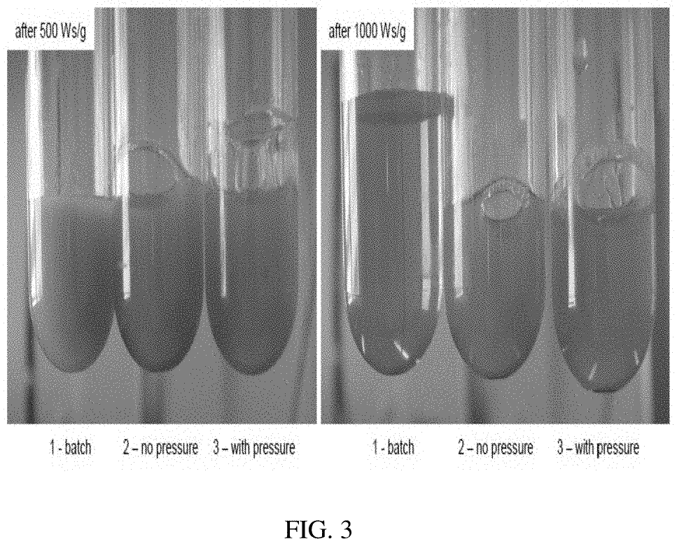

FIG. 3 comprises photographs illustrating the appearance of Series 1, 2 and 3 after energy level of 500 Ws/g and 1000 Ws/g. In series 2, sonication was performed in discrete recirculation without pressure. In series 3, a pressure of 1.5 to 2 bar(g) was applied. This resulted in a higher power output, and a higher sonication. Intensity, faster treatment time and also faster temperature increases were observed. Interestingly, after 500 Ws/g, sample 3 that was produced in recirculation under pressure, appeared most translucent. After 1000 Ws/g, a clear difference between sample 2 and 3 was observed, as the sample sonicated without pressure (Series 2) was still more dull. However, sample 3 did not seem to be on the level of the batch sample at this specific energy input.

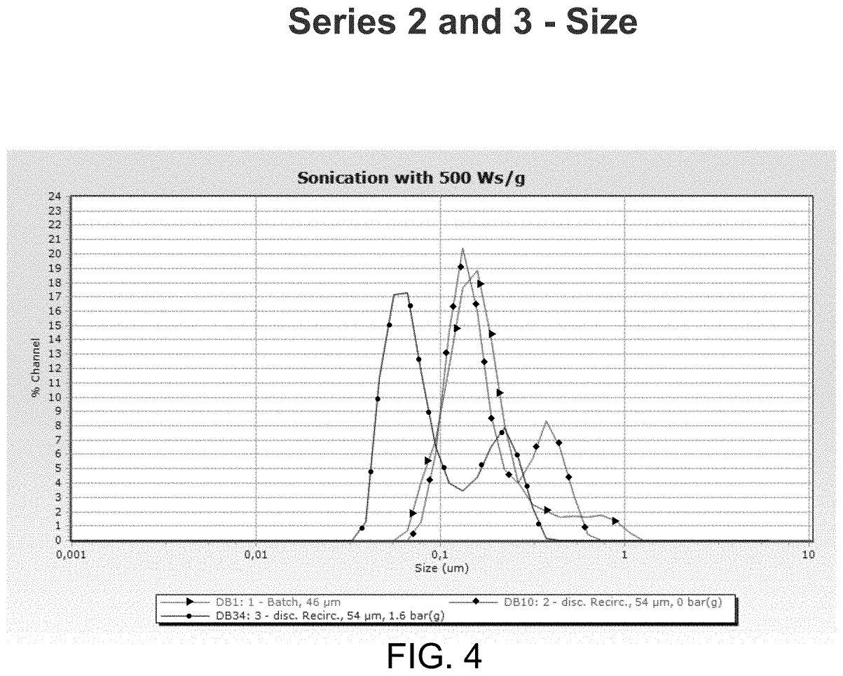

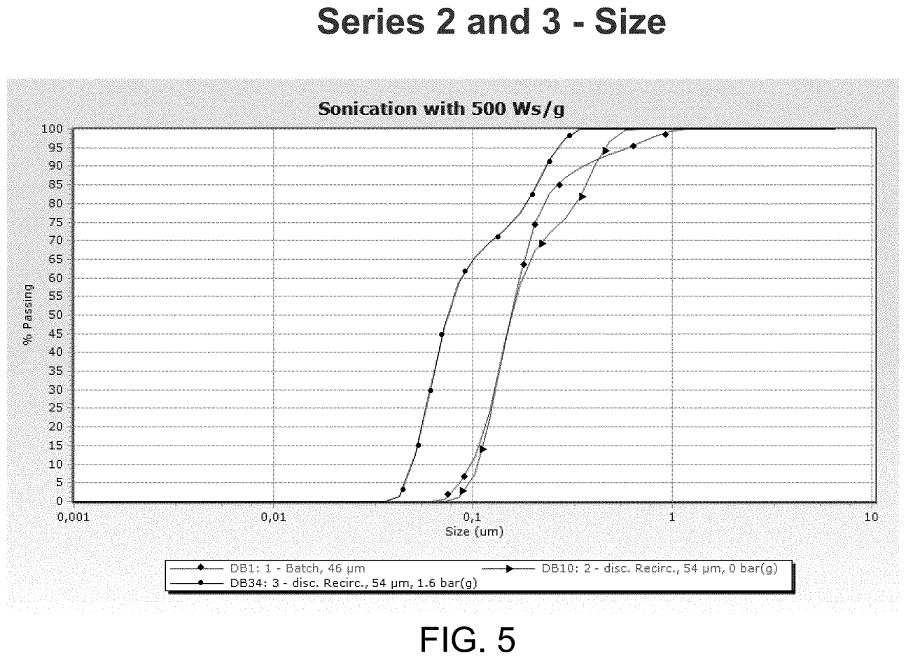

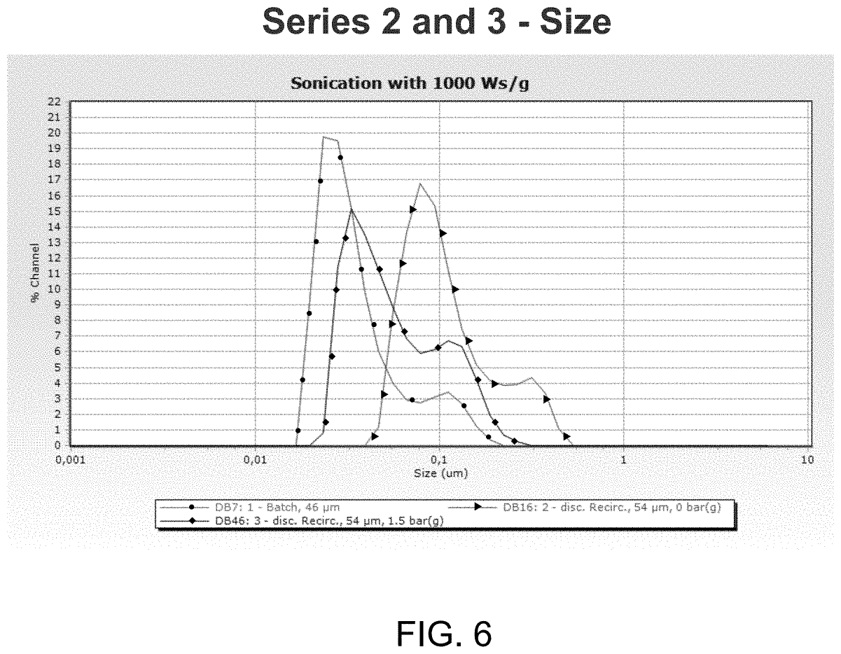

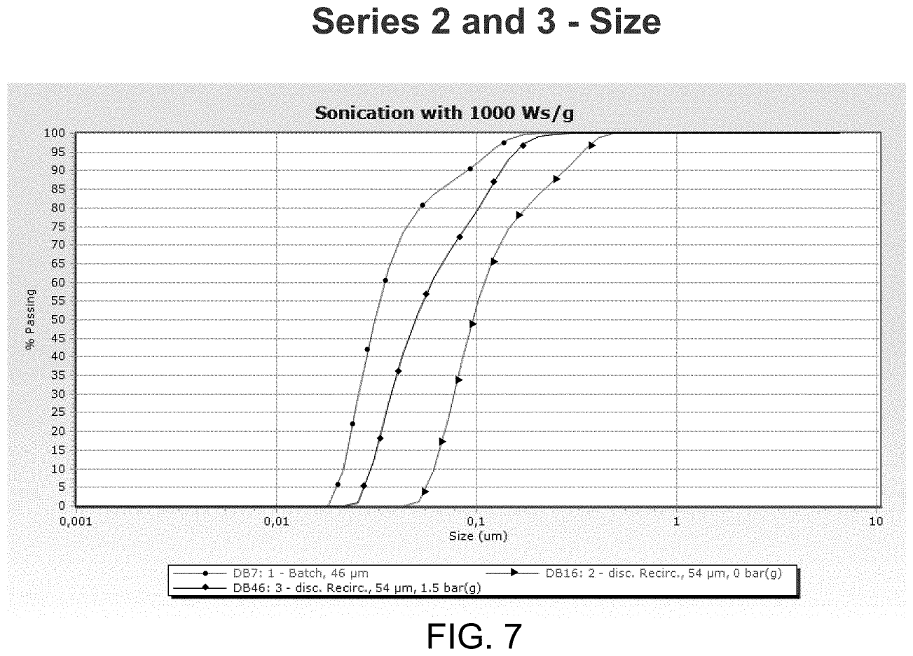

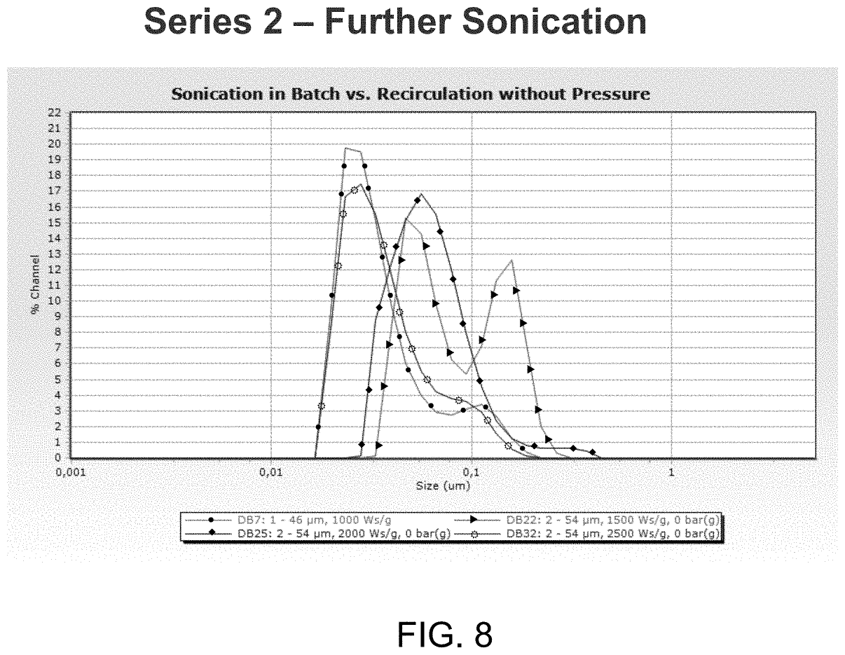

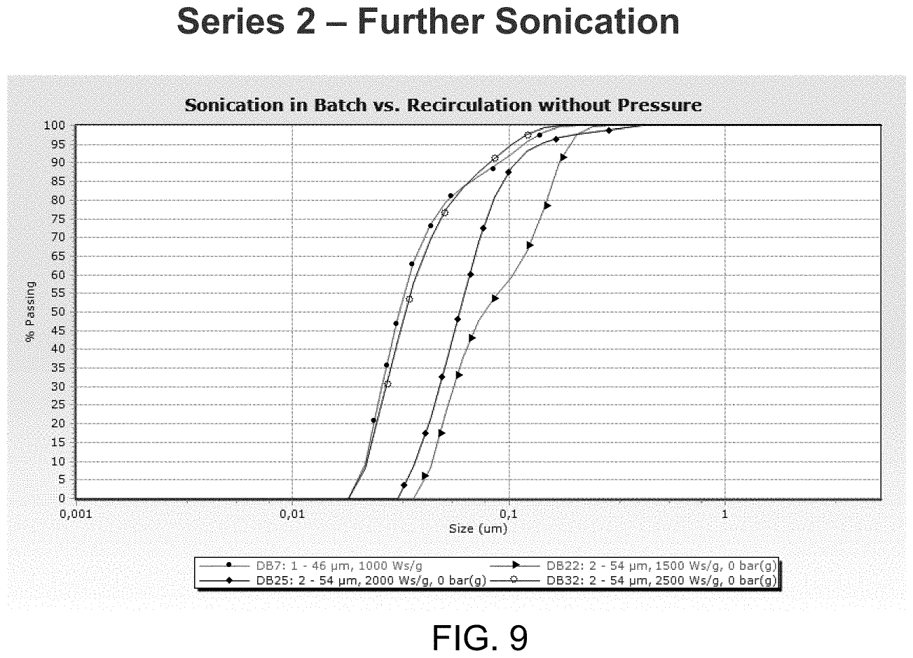

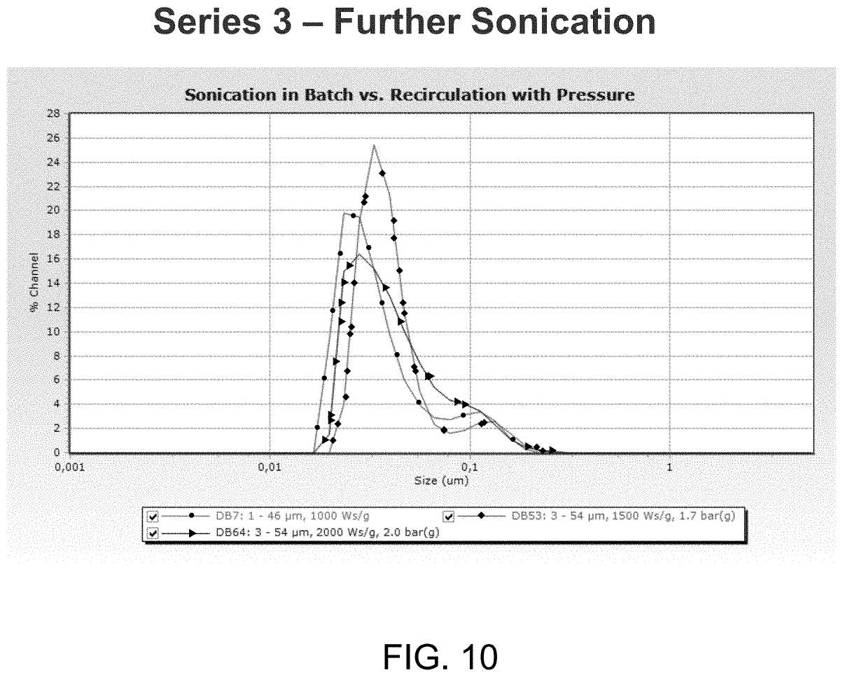

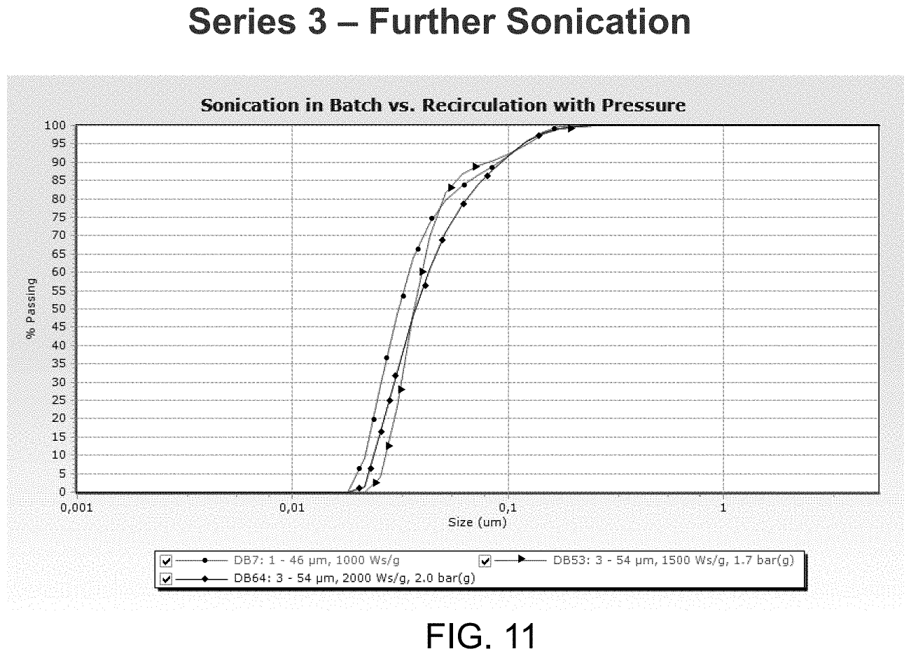

FIG. 4 illustrates a droplet size of diluted samples Series (1, 2 and 3) after energy level of 500 Ws/g. The optical trend was confirmed by particle size measurement. After 500 Ws/g, sample 3 had the smallest particle size distribution, with an average droplet size of 111 nm. Sample 2 was, with 210 nm, in the range of the batch sample (1: 205 nm). FIG. 5 illustrates a Percentage Pass of diluted samples Series (1, 2 and 3) at energy level of 500. FIG. 6 illustrates a droplet size of diluted samples Series (1, 2 and 3) at energy level of 1000. Indeed, after 1000 Ws/g sample 3 had a much smaller droplet size distribution than sample 2, with an average of 67 nm compared to 130 nm. It did not meet the level of sample 1 (43 nm). FIG. 7 is an illustration of the percentage pass of diluted samples Series (1, 2 and 3) at energy level of 1000. FIG. 8 is an illustration of droplet size of diluted samples Batch vs. Recirculation without Pressure at different energy levels. To reach the level of the batch sample, 2500 Ws/g had to be invested when sonicating in recirculation without pressure. FIG. 9 is an illustration of percentage pass of diluted samples Batch vs. Recirculation without Pressure at different energy levels. FIG. 10 is an illustration of droplet size of diluted samples Batch vs. Recirculation with Pressure at different energy levels. With pressure, 1500 Ws/g were sufficient to reach a mean droplet size of 46 nm. Further sonication changed the droplet size only marginally. FIG. 11 is an illustration of the Percentage Pass of diluted samples Batch vs. Recirculation with Pressure at different energy levels.

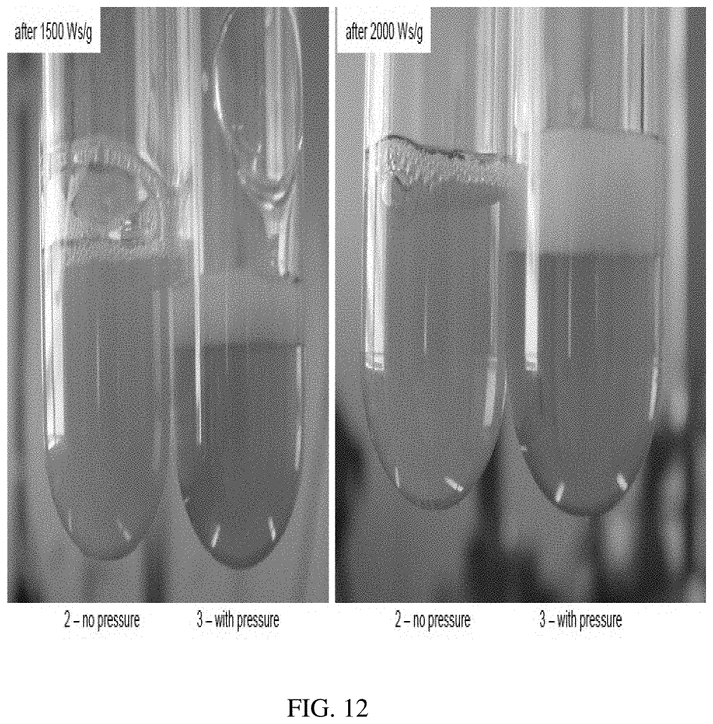

FIG. 12 are photographs illustrating the appearance of Series 2 and 3 with and without pressure at energy level of 1500 Ws/g and 2000 Ws/g. The benefit of pressure was apparent when directly comparing samples at the respective specific energy inputs. The samples sonicated without pressure appeared slightly more dull, whilst those sonicated with pressure were clear and more translucent.

The circulation constellation system in Series (2) and (3) yielded an amplitude of as shown in FIGS. 4-11. The setup included a progressive cavity pump MD012-12 (Seepex), pressure- and thermo-sensor, pressure valve and cooling tubes. The trials were conducted as discrete recirculation (catching sonicated material in a separate vessel, to avoid mixing with unsonicated liquid). The Droplet size of diluted samples was measured with a Nano-Flex® by Colloid Metrix (Dynamic Light Scattering). It decreased with increasing specific energy input.

Results: In batch, a mean droplet size of 43 nm was achieved with a specific energy input of 1000 Ws/g. In discrete recirculation with pressure of 1.5 to 2.0 bar(g), 1500 Ws/g were sufficient to reach this level. The objective, droplets of about 50 nm, was reached after 1000 Ws/g in series (3). Without pressure, 2500 Ws/g had to be invested to reach an average droplet size of 43 nm; the objective was met after 2000 Ws/g. By pressure, the power output of the device is increased (here 675 to 1500 W). This results in a higher sonication intensity (40 vs. 88 W/cm²), as well as a faster production time, as desired energy inputs are reached faster (28 min vs. 12 min to reach 1000 Ws/g). Due to the higher power under pressure, temperature increases faster; therefore, stronger cooling needs to be applied.

### Example 2

The objective of this example was to compare the nanoemulsion properties of synthetic surfactant (Tween 80) with natural surfactant Saponins by using the same formulation and method described in Example 1. The quantity of other ingredients is according to Table 2. The droplet size and translucency was comparable to Example 1.

TABLE 2

| Component | % wt/wt |
|---|---|
| CBD Isolate Powder (99% pure) | 0.5%-5% |
| Organic Deodorized Oleic Sunflower Oil | 0.5%-5% |
| Oil Lemon Type 10-fold Extra | 0%-2% |
| Tween 80 | 2%-5% |
| Alcolec LEM (Enzyme-Modified Soy Lecithin) | 2%-5% |
| Tocopherol Mix (Fortium MTD10) | 0%-0.5% |
| Vegetable Glycerin | 0%-30% |
| Crystalline Sorbitol | 0%-30% |
| Deionized water | To 100% |

### Example 3

Nanoemulsion was prepared according to the method described for Example 1 with the difference that the chitosan solution was also added and resulting nanoemulsion was filtered through 220 nm filter. The quantity of other ingredients is according to Table 3. The droplet size and translucency was comparable to Example 1.

TABLE 3

| Component | % wt/wt |
|---|---|
| CBD Isolate Powder (99% pure) | 0.5%-5% |
| Organic Deodorized Oleic Sunflower Oil | 0.5%-5% |
| Oil Lemon Type 10-fold Extra | 0%-2% |
| Chitosan Solution (3% chitosan) | 0%-2% |
| Saponin DAB 9 (Base Quillaja) | 2%-10% |
| Alcolec LEM (Enzyme-Modified Soy Lecithin) | 2%-5% |
| Tocopherol Mix (Fortium MTD10) | 0%-0.5% |
| Vegetable Glycerin | 0%-30% |
| Crystalline Sorbitol | 0%-30% |
| Deionized water | To 100% |

Results: Bio-absorption was evaluated indirectly using Nitric Oxide as a surrogate biomarker by comparing the release of nitric oxide in salvia (shown in Table 4). As it is evident in the table below, the onset time is as quick as 5 min; and the table illustrates the results of comparative Nitric oxide levels (as a biomarker surrogate for cannabidiol “CBD” absorption) after taking 10 mg CBD in nanoemulsion form in water and measured using “Berkeley Fit Nitric Oxide Test with saliva Nitric Oxide Test Strips”

TABLE 4

| | TIME | NITRIC OXIDE |
|---|---|---|
| No | (MIN) | LEVEL IN SALIVA |
| 1 | 0 | 20 (BASE LINE) |
| 2 | 5 | 30 |
| 3 | 10 | 40 |
| 4 | 15 | 60 |
| 5 | 20 | 40 |
| 6 | 30 | 60 |
| 7 | 60 | 110 |
| 8 | 90 | 20 |

### INDEX OF ELEMENTS

The following Table 5 lists variations in ingredients for exemplary embodiments of the present invention.

TABLE 5

| Composition Ingredient | Types of Source Material |
|---|---|
| Saponins | Plant Source |
| | Quillaja Saponin |
| Phospholipids | Natural |
| | Synthetic |
| | Lecithin |
| | Phospholipid |
| Lipids And Fatty Acids | Long Chain Fatty Acids |
| | Medium Chain Fatty Acids |
| | Mono, Di, And Tri Glyceride |
| Sugar Alcohols | Sorbitol |
| Simple Polyols | Glycerol |
| Optionally Synthetic | |
| Surfactants | |
| Anti-oxidants | Natural |
| | Synthetic |
| Preservatives | Sorbic Acid And Salts |
| | Benzoic Acid And Salt |
| | Natural |
| | Synthetic |
| Terpenes And Terpenoids | Citral |
| | Limonene |
| | Lemon oil |
| Piperine | |
| Chitosan | |

### CONCLUSION

Unless otherwise defined, all technical and scientific terms used herein have the same meaning as commonly understood by one of ordinary skill in the art to which this invention belongs. Although methods and materials similar to or equivalent to those described herein can be used in the practice or testing of the nanoemulsion and nanoparticle composition using saponins and method for increasing bioavailability, suitable methods and materials are described above. All publications, patent applications, patents, and other references mentioned herein are incorporated by reference in their entirety to the extent allowed by applicable law and regulations. The nanoemulsion and nanoparticle composition using saponins and method for increasing bioavailability may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

It will be appreciated that the methods and compositions of the present disclosure can be incorporated in the form of a variety of embodiments, only a few of which are disclosed herein. It will also be apparent for the expert skilled in the field that other embodiments exist and do not depart from the spirit of the invention. Thus, the described embodiments are illustrative and should not be construed as restrictive.

Accordingly, the preceding exemplifications merely illustrate the principles of the various embodiments. It will be appreciated that those skilled in the art will be able to devise various arrangements which, although not explicitly described or shown herein, embody the principles of the embodiments and are included within its spirit and scope. Furthermore, all examples and conditional language recited herein are principally intended to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the various embodiments, therefore, is not intended to be limited to the exemplary embodiments shown and described herein.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it unless it is contextually clear either one of the elements or more than one of the elements is described. The term “about” as used herein refers to a value within 10% of the underlying parameter (i.e., plus or minus 5%), and use of the term “about” at the beginning of a string of values modifies each of the values (i.e., “about 1, 2 and 3” refers to about 1, about 2 and about 3). Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

As used herein, the term “substantially” refers to approximately the same shape or value as stated as recognized by one of ordinary skill in the art.

While several embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of embodiments.

Trademarks: the product names used in this document are for identification purposes only; and are the property of their respective owners.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the disclosure or of any exemplified term. Likewise, the disclosure is not limited to various embodiments given in this specification.

Unless otherwise defined, all technical and scientific terms used herein have the same meaning as commonly understood by one of ordinary skill in the art to which this disclosure pertains. In the case of conflict, the present document, including definitions will control.

### LIST OF REFERENCES CITED

The following is a list of references cited in this application. All of these citations are hereby incorporated by reference.
[1] Y. Yang, M. E. Leser, A. A. Sher, and D. J. McClements, “Formation and stability of emulsions using a natural small molecule surfactant: Quillaja saponin (Q-Naturale®),” Food Hydrocolloids, vol. 30, no. 2, pp. 589-596, March 2013.
[2] S. Yalçnöz and E. Erçelebi, “Potential applications of nano-emulsions in the food systems: an update,” Mater. Res. Express, vol. 5, no. 6, p. 062001, June 2018.
[3] M. Schultz and V. Monnier, “Composition and method for manufacturing clear beverages comprising nanoemulsions with quillaja saponins,” US20150030748A1, Jan. 29, 2015.
[4] Ozaki S., Sasakura H., Yokoyama M., “O/w-type emulsified composition of liposoluble material,” JP2010142205A, Jul. 1, 2010.
[5] D. Schrader, C. Homner, and C. SABATER-LÜNTZEL, “Compositions with a surfactant system comprising saponins, and lecithin,” WO2011089249A1, Jul. 28, 2011.
[6] T. Riess, C. SABATER-LÜNTZEL, C. Homner, and D. Schrader, “Solubilization agent for solubilizing polyphenols, flavonoids and/or diterpenoid glucosides,” EP2359702B1, Jun. 4, 2014.
[7] J. Rao and D. J. McClements, “Impact of lemon oil composition on formation and stability of model food and beverage emulsions,” Food Chemistry, vol. 134, no. 2, pp. 749-757, September 2012.
[8] Q. Choudhry et al., “Saponin-Based Nanoemulsification Improves the Antioxidant Properties of Vitamin A and E in AML-12 Cells,” IJMS, vol. 17, no. 9, p. 1406, August 2016.
[9] K. Holmberg, “Natural surfactants,” Current Opinion in Colloid & Interface Science, vol. 6, no. 2, pp. 148-159, May 2001.
[10] P. Kumar Gupta et al., “An Update on Nanoemulsions Using Nanosized Liquid in Liquid Colloidal Systems,” in Nanoemulsions—Properties, Fabrications and Applications, K. Seng Koh and V. Loong Wong, Eds. IntechOpen, 2019.
[11] A. Teo, K. K. T. Goh, and S. J. Lee, “Nanoparticles and Nanoemulsions,” in Functional Foods and Dietary Supplements, A. Noomhorm, I. Ahmad, and A. K. Anal, Eds. Chichester, UK: John Wiley & Sons, Ltd, 2014, pp. 405-435.
[12] D. J. McClements, “Nanoemulsions versus microemulsions: terminology, differences, and similarities,” Soft Matter, vol. 8, no. 6, pp. 1719-1729, January 2012.
[13] T. J. Wooster, M. Golding, and P. Sanguansri, “Impact of Oil Type on Nanoemulsion Formation and Ostwald Ripening Stability,” Langmuir, vol. 24, no. 22, pp. 12758-12765, November 2008.
[14] L. Hu, Z. Mao, and C. Gao, “Colloidal particles for cellular uptake and delivery,” J. Mater. Chem., vol. 19, no. 20, pp. 3108-3115, May 2009.
[15] “Drug Bioavailability—Clinical Pharmacology,” Merck Manuals Professional Edition. (accessed Jun. 8, 2020).
[16] P. A. Lachance and Y. T. Das, “1.11—Nutraceuticals,” in Comprehensive Medicinal Chemistry II, J. B. Taylor and D. J. Triggle, Eds. Oxford: Elsevier, 2007, pp. 449-461.
[17] S. V. Gupta and Y. V. Pathak, Advances in Nutraceutical Applications in Cancer: Recent Research Trends and Clinical Applications. CRC Press, 2019.
[18] “Cannabinoids—Alcohol and Drug Foundation.” Australia, (accessed Jun. 8, 2020).
[19] E. Bombardelli, G. F. Patri, and R. Pozzi, “Complexes of saponins with phospholipids and pharmaceutical and cosmetic compositions containing them,” EP0283713A2, Sep. 28, 1988.
[20] D. Kregiel et al., “Saponin-Based, Biological-Active Surfactants from Plants,” in Application and Characterization of Surfactants, R. Najjar, Ed. InTech, 2017.
[21] J. Li et al., “A review on phospholipids and their main applications in drug delivery systems,” Asian Journal of Pharmaceutical Sciences, vol. 10, no. 2, pp. 81-98, April 2015.
[22] D. J. McClements and H. Xiao, “Potential biological fate of ingested nanoemulsions: influence of particle characteristics,” Food Funct., vol. 3, no. 3, pp. 202-220, 2012.
[23] Y. Singh et al., “Nanoemulsion: Concepts, development and applications in drug delivery,” Journal of Controlled Release, vol. 252, pp. 28-49, April 2017.
[24] C.-P. Liang, M. Wang, J. E. Simon, and C.-T. Ho, “Antioxidant activity of plant extracts on the inhibition of citral off-odor formation,” Molecular Nutrition & Food Research, vol. 48, no. 4, pp. 308-317, 2004.
[25] E. M. Persson et al., “A clinical single-pass perfusion investigation of the dynamic in vivo secretory response to a dietary meal in human proximal small intestine,” Pharm. Res., vol. 23, no. 4, pp. 742-751, April 2006.
[26] F. Kuo, B. Subramanian, T. Kotyla, T. A. Wilson, S. Yoganathan, and R. J. Nicolosi, “Nanoemulsions of an anti-oxidant synergy formulation containing gamma tocopherol have enhanced bioavailability and anti-inflammatory properties,” Int J Pharm, vol. 363, no. 1-2, pp. 206-213, November 2008.
[27] A. H. Shojaei, “Buccal Mucosa as a Route for Systemic Drug Delivery.” J Pharm Pharmaceut. Sci., 1 (1):15-30, 1998
[28] W. R. Galey, H. K. Lonsdale, and S. Nacht, “The in vitro permeability of skin and buccal mucosa to selected drugs and tritiated water,” J. Invest. Dermatol., vol. 67, no. 6, pp. 713-717, December 1976.
[29] D. Harris and J. R. Robinson, “Drug delivery via the mucous membranes of the oral cavity,” J Pharm Sci, vol. 81, no. 1, pp. 1-10, January 1992.
[30] L. P. J, D. B, and R. R. N, “Rectal drug delivery: A promising route for enhancing drug absorption,” Asian Journal of Research in Pharmaceutical Sciences, vol. 2, no. 4, pp. 143-149, December 2012.
[31] N. J. Alexander, E. Baker, M. Kaptein, U. Karck, L. Miller, and E. Zampaglione, “Why consider vaginal drug administration?,” Fertility and Sterility, vol. 82, no. 1, pp. 1-12, July 2004.
[32] S. Türker, E. Ózer, and Y. Ozer, “Nasal route and drug delivery systems,” Pharm World Sci, vol. 26, no. 3, pp. 137-142, June 2004.
[33] M. Kumar, A. Misra, A. K. Babbar, A. K. Mishra, P. Mishra, and K. Pathak, “Intranasal nanoemulsion based brain targeting drug delivery system of risperidone,” Int J Pharm, vol. 358, no. 1-2, pp. 285-291, June 2008.
[34] R. C. Kaufman, “Nanoparticle compositions and methods as carriers of nutraceutical factors across cell membranes and biological barriers,” U.S. Pat. No. 9,925,149 B2, issued Mar. 27, 2018.

What is claimed is:

1. A nanoemulsion composition comprising:
an all natural bioactive compound at about 0.1% wt/wt to about 50% wt/wt, comprising plant and/or food active ingredients without synthetic ingredients comprising: cannabinoids, nutraceuticals, vitamins, or any combination thereof;
one or more natural surfactant(s), comprising saponins and/or phospholipids;
wherein said composition comprises nanoemulsions with about 43 nanometers to about 50 nanometers in diameter or droplet size;
wherein said composition is water-soluble and able to increase a bioavailability of the bioactive compound as compared to a composition comprising only synthetic surfactants; and
wherein said composition is able to safely be consumed by humans in food and beverage preparations, supplements, medicines, pet foods, skin care products, cosmetics, personal care products and hygiene products.

2. The composition of claim 1, wherein said natural surfactant comprises up to about 25% wt/wt of powdered saponin extract and/or up to about 50% wt/wt of liquid saponin extract.

3. The composition of claim 2, wherein said natural surfactant comprises about 2.5% by weight/weight of powder or liquid saponin extracted from quillaia Saponins.

4. The composition of claim 1, wherein said natural surfactant comprises about 2.5% by weight/weight of a phospholipid derived from an enzyme modified lecithin.

5. The composition of claim 1, wherein said natural surfactant comprises: about 2.5% by weight/weight of pure saponin extracted from quillaia Saponins, and about 2.5% by weight/weight of a phospholipid derived from enzyme modified lecithin.

6. The composition of claim 1, wherein the composition further comprises Piper nigrum up to about 0.5% wt/wt and that is able to increase bioavailability and to enhance bioefficacy of said composition.

7. The composition of claim 1, wherein the composition further comprises chitosan up to about 5% wt/wt and able to improve said composition's absorption.

8. The composition of claim 1, further comprising a co-surfactant comprising glycerine or sorbital, up to about 5% wt/wt.

9. The composition of claim 1, further comprising a Terpenes and/or Terpenoids up to about 10% wt/wt.

10. A method of making a nanoemulsion composition comprising:
a) combining a pre-emulsion composition comprising the ingredients of:
an all natural bioactive compound at about 0.1% wt/wt to about 50% wt/wt, comprising plant and/or food active ingredients without synthetic ingredients comprising: cannabinoids, nutraceuticals, vitamins, or any combination thereof;
one or more natural surfactant(s), comprising saponins and/or phospholipids;
b) homogenizing said pre-emulsion composition using a high shear mixer about 5 minutes at about 15,000 rpms;
c) sonicating the pre-emulsion composition into a nanoemulsion composition using one or more methods comprising:
a batch sonification at an energy level of 1000 Ws/g;
sonification at an energy level of about 2000 Ws/g to about 2500 Ws/g, and without pressure while recirculating the pre-emulsion composition;
sonification at an energy level of about 1500 Ws/g, with applying a high pressure of about 1.5 to about 2.0 bar (g), while recirculating the pre-emulsion composition;
d) wherein said composition comprises nanoemulsions with about 43 nanometers to about 50 nanometers in diameter or droplet size;
e) wherein said composition is water-soluble and able to increase a bioavailability of the bioactive compound as compared to a composition comprising only synthetic surfactants; and
f) wherein said composition is able to safely be consumed by humans in food and beverage preparations, supplements, medicines, pet foods, skin care products, cosmetics, personal care products and hygiene products.

11. The composition of claim 10, wherein said natural surfactant comprises up to about 25% wt/wt of powdered saponin extract and/or up to about 50% wt/wt of liquid saponin extract.

12. The composition of claim 11, wherein said natural surfactant comprises about 2.5% by weight/weight of powder or liquid saponin extracted from quillaia Saponins.

13. The composition of claim 10, wherein said natural surfactant comprises about 2.5% by weight/weight of a phospholipid derived from an enzyme modified lecithin.

14. The composition of claim 10, wherein said natural surfactant comprises: about 2.5% by weight/weight of pure saponin extracted from quillaia Saponins, and about 2.5% by weight/weight of a phospholipid derived from enzyme modified lecithin.

15. The composition of claim 10, wherein the composition further comprises Piper nigrum up to about 0.5% wt/wt and that is able to increase bioavailability and to enhance bioefficacy of said composition.

16. The composition of claim 10, wherein the composition further comprises chitosan up to about 5% wt/wt and able to improve said composition's absorption.

17. The composition of claim 10, further comprising a co-surfactant comprising glycerine or sorbital, up to about 5% wt/wt.

18. The composition of claim 10, further comprising a Terpenes and/or Terpenoids up to about 10% wt/wt.
